(19) United States
(12) Patent Application Publication
(10) Pub. No.: US 2007/0112043 A1
(43) Pub. Date: May 17, 2007

(54) Acylated and non-acylated imidazo[2,1-b]-1,3,4,-thiadiazole-2-sulfonamides, and uses thereof

(76) Inventors: James B. Jaquith, Pincourt (CA); John W. Gillard, Baie D'Urfe (CA)

Correspondence Address: AEGERA THERAPEUTICS INC., 810 CHEMIN DU GOLF, VERDUN, QC, H3E-1A8, CA

(21) Appl. No.: 10/559,675

(22) PCT Filed: Jun. 14, 2004
(86) PCT No.: PCT/CA04/00873

§ 371 Date: Dec. 6, 2005

Related U.S. Application Data
(60) Provisional application No. 60/477,967, filed on Jun. 13, 2003.

(51) Int. Cl. A61K 31/433; C07D 487/04

(52) U.S. Cl. 514/362; 548/126

(57) ABSTRACT

This invention relates to novel compounds of Formula (I): and the use of compounds of Formula (I) in the treatment of neuronal disorders of the central and peripheral nervous systems and for the treatment of proliferative diseases, such as cancer.

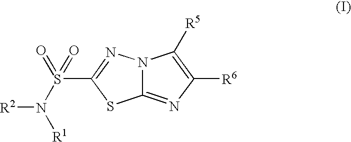

### FIELD OF THE INVENTION

This invention relates to imidazo-thiadiazole-sulfonamide compounds useful in the treatment of neuronal disorders of the central and peripheral nervous systems and in the treatment of proliferative diseases, such as cancer and inflammation.

### BACKGROUND OF THE INVENTION

The Applicant has previously demonstrated that selected compounds represented by Formula I, 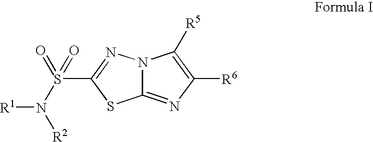
wherein R¹ and R² are independently H or C(1-4) alkyl, protect SCG neurons from several neurotoxic insults, including NGF withdrawal and treatment with chemotherapeutics such as Taxol™ and cisplatin. When such agents are administered to rats treated with Taxol™, either during or after a two week dosing period, marked improvements are observed in the animal's general health, weight gain, gait, and nerve conductance as compared to animals treated with Taxol™ alone (PCT Application No. CA02/01942 (WO 03/051890)). Compounds from this class also aid in the regeneration of neurons damaged as a result of sciatic nerve crush and protect retinal ganglion neurons from ocular stroke. Additionally, cortical motor neurons are protected from malonate induced death (PCT Application No. CA02/01942 (WO 03/051890)).

Other uses of select compounds represented by formula I in which R¹═R²═H include anti-bacterial agents (Gadad, A. K. Eur J. Med. Chem., 35(9), 853-857, 2000) and carbonic anhydrase (CA) inhibitors (Barnish, I. T., et al., J. Med. Chem., 23(2), 117-121, 1980; Barnish, I. T. et al GB 1464259, abandoned; Supuran, C, T. Met.-Based Drugs 2(6), 331-336, 1995). The Applicant has demonstrated that the introduction small alkyl groups at R¹ and R² dramatically reduce the CA activity of these compounds, while maintaining their neuronal protection in vitro (PCT Application No. CA02/01942 (WO 03/051890)).

One specific compound from this class, namely 5-Bromo-6-phenylmidazo[2,1-b]-1,3,4,-thiadiazole-2-sulfonamide (R¹═R²═H, R⁵═Br, R⁶═Ph, abbreviated herein as 5-Br-6-Ph-ITS), has been shown to display anti-proliferative activity (Gadad, A. K. India. Arzneim.-Forsch., 49(10), 858-863, 1999). However, this compound is not an attractive therapeutic agent, due to the active bromine at C5. Furthermore, the Applicant has demonstrated that this compound is rapidly degraded in microsomal fractions, limiting its therapeutic potential.

Prodrugs are precursors of active forms of a drug, which degrade into the active form in vivo. The use of simple N-C(1-4)acylsulfonamides as prodrugs has been previously described for the COX-2 inhibitors parecoxib sodium and celecoxib (Talley, J. J., et. al., J. Med. Chem. 2000 May 4; 43(9):1661-3 and Mamidi, R. N., et al. Biopharm. Drug Dispos. 2002 October; 23(7):273-82).

### SUMMARY OF THE INVENTION

The present invention relates to imidazo[2,1-b]-1,3,4,-thiadiazole-2-sulfonamides, represented by Formula I:

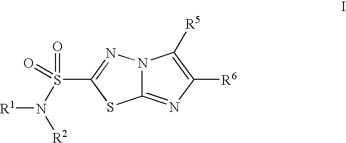

In particular, this application is concerned with N-acyl sulfonamides, wherein R¹ is represented by an acyl group (formula I-b), and the use of such compounds for the treatment of neurodegenrative diseases and for the treatment of proliferative diseases. The application is also concerned with the use of sulfonamides, wherein R¹ and R² independently represent H or (C1-4) alkyl (formula I-a), for the treatment of proliferative diseases.

The N-acylsulfonamides represented by formula I-b display altered solubility and pharmacokinetic properties as compared to their parent sulfonamides, formula I-a. This may be characterized by aqueous soluble formulations with neutral pH and/or improved pharmacokinetics.

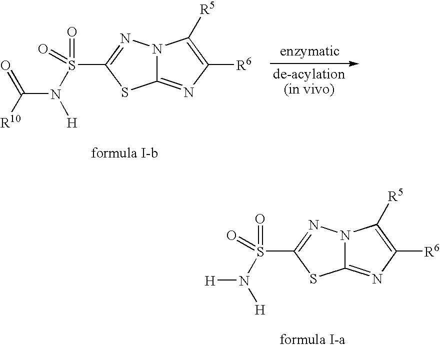

Compounds represented by formula I-b are converted in vivo to their parent sulfonamides and may act as prodrugs for the parent sulfonamide.

### BRIEF DESCRIPTION OF THE FIGURE

FIG. 1 shows the effects of Compounds 14, 45, 39 and 31 on Cisplatin-Induced Attenuation of SNCV. Rats treated with cisplatin display a reduced maturational increase in SNCV as compared to control animals. This loss in SNCV is prevented by treatment with compound 14 (10 mg/kg). The N-acyl derivatives 45, 39, and 31 (3, 10 and 30 mg/kg), demonstrating similar potency at 30 mg/kg in this model of peripheral neuropathy.

### DETAILED DESCRIPTION OF THE INVENTION AND PREFERRED EMBODIMENTS

The imidazo[2,1-b]-1,3,4,-thiadiazole-2-sulfonamides of the present invention are represented by Formula I: 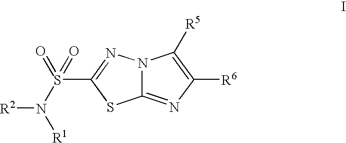
or a pharmaceutically acceptable salts thereof, wherein:

R¹ and R² are individually selected from the group consisting of:
a) H and C(1-4)-alkyl;
b) C(O)R⁹, wherein R⁹ is selected from C(1-18) substituted or unsubstituted alkyl, substituted or unsubstituted aryl, substituted or unsubstituted heteroaryl; and
c) C(O)—(CH₂)ₙ—(C(O))ₚ—(OCH₂CH₂)ₘOR¹⁰, wherein n=0-6, p=0-1, m=0-22, and R¹⁰ is H, substituted or unsubstituted C(1-6) alkyl, substituted or unsubstituted aryl, substituted or unsubstituted heteroaryl;
d) C(O)—(CHR¹¹)ₙ—NR²R¹³, wherein n=1-5, R¹¹ is selected from the group consisting of hydrogen, substituted or unsubstituted C(1-8) alkyl, substituted or unsubstituted C(1-8) aralkyl, substituted or unsubstituted C(1-8) aryl, substituted or unsubstituted C(1-8) heteroaryl, and R¹² and R¹³ are individually selected from the group consisting of hydrogen, substituted or unsubstituted C(1-8) alkyl, substituted or unsubstituted C(1-8) aralkyl, substituted or unsubstituted C(1-8) aryl, substituted or unsubstituted C(1-8) heteroaryl, substituted or unsubstituted C(1-8) alkylcarbonyl, substituted or unsubstituted C(1-8) arylcarbonyl, substituted or unsubstituted C(1-8) heteroarylcarbonyl, or wherein R¹² and R¹³ are combined to for members of a 5 to 7 membered substituted or unsubstituted heterocyclic ring system;

R⁵ is selected from the group consisting of H, methyl, and substituted or unsubstituted benzyl

R⁶ is selected from the group consisting of

(i) fluoro C(1-6)-alkyl, substituted and unsubstituted C(6-16)-aryl, substituted and unsubstituted heteroaryl, substituted and unsubstituted biphenyl, substituted and unsubstituted diphenyl ether, substituted and unsubstituted coumarinyl, and adamantyl;

wherein adjacent carbons in ring systems of the aryl or heteroaryl R⁵ substituents or adjacent carbons in ring systems of the aryl, heteroaryl, biphenyl, diphenyl ether, or coumarinyl R⁶ substituents may together be substituted by a fused cycloalkyl or heterocycloalkyl ring, which cycloalkyl or heterocycloalkyl ring may be further substituted by one or more an alkyl groups, or two alkyl groups joined to form a ring;

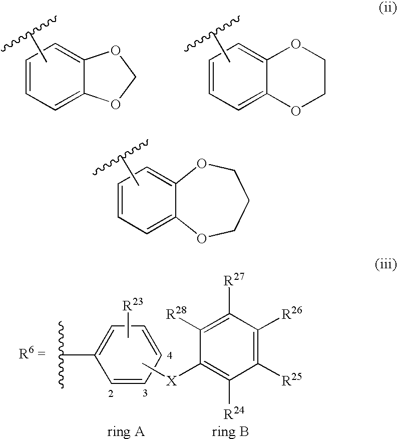

wherein

X is represented by a bond, O or S(O)ₙ, wherein n=0, 1, or 2, and is attached to ring A at the 2, 3, or 4 position;

R²³ on ring A is selected from the group consisting of H, halogen, C(1-8)alkyl, C(1-8) alkoxy and represents up to 4 substitutions;

R²⁴ through R²⁸ of ring B is independently selected from the group consisting of H, halogen, C(1-8) alkyl, C(1-8) flouroalkyl, C(1-8) alkoxy,

wherein any two adjacent R groups may be combined to form members of a fused aryl, substituted aryl, heteroaryl, or substituted heteroaryl, ring system; and 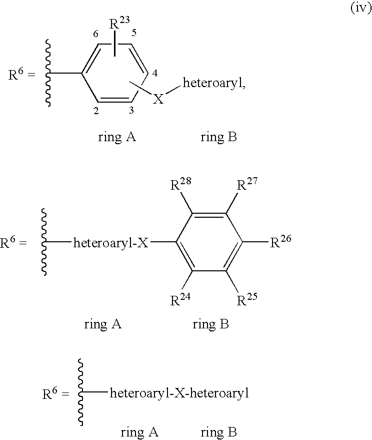
wherein

X is represented by a bond, O or S(O)ₙ, wherein n=0, 1, or 2;

R²³ on ring A is selected from the group consisting of H, halogen, C(1-8) alkyl, C(1-8) alkoxy and represents up to 4 substitutions;

the heteroaryl ring systems of ring A and B contain at least on heteroatom and are substituted or unsubstituted;

R²⁴ through R²⁸ of ring B is independently selected from the group consisting of H, halogen, C(1-8) alkyl, C(1-8) flouroalkyl, C(1-8) alkoxy; and

wherein any two adjacent R groups may be combined to form members of a fused aryl, substituted aryl, heteroaryl, or substituted heteroaryl, ring system.

In the definitions of the groups of Formula I, C(1-8) alkyl means a straight-chain or branched alkyl group having 1 to 8 carbon atoms, such as methyl, ethyl, propyl, iso-propyl, n-butyl, iso-butyl, sec-butyl, tert-butyl, pentyl, iso-amyl, neopentyl, 1-ethylpropyl, hexyl, and octyl. The C(1-8) alkyl moiety of C(1-8) alkoxy, C(1-8) alkylsulfonyl, C(1-8) alkoxylcarbonyl, C(1-8) alkylaminocarbonyl has the same meaning as C(1-8) alkyl defined above. The acyl moiety of the acyl and the acyloxy group means a straight-chain or branched alkanoyl group having 1 to 18 carbon atoms, such as acetyl, propanoyl, butyryl, valeryl, pivaloyl and hexanoyl, and arylcarbonyl group described below, or a heteroarylcarbonyl group described below. The aryl moiety of the aryl, the arylcarbonyl and arylaminocarbonyl groups means a group having 6 to 16 carbon atoms such as, but not limited to, phenyl, biphenyl, naphthyl, or pyrenyl. The heteroaryl moiety of the heteroaryl and the heteroarylcarbonyl groups contain at least one hetero atom from O, N, and S, such as, but not limited to pyridyl, pyrimidyl, pyrroleyl, furyl, benzofuryl, thienyl, benzothienyl, imidazolyl, triazolyl, quinolyl, iso-quinolyl, benzoimidazolyl, thiazolyl, benzothiazolyl, oxazolyl, and indolyl. The aralkyl moiety of the aralkyl and the aralkyloxy groups having 7 to 15 carbon atoms, such as, but not limited to, benzyl, phenethyl, benzhydryl, and naphthylmethyl. The heteroaralkyl moiety of the heteroaralkyl and the heteroaralkyloxy groups having 7 to 15 carbon such as, but not limited to, pyridylmethyl, quinolinylmethyl, and iso-quinolinylmethyl. The substituted C(1-8) alkyl group has 1 to 3 independently-substitutuents, such as but not limited to hydroxyl, C(1-8) alkyloxy, C(1-8) alkylthio, carboxyl, C(1-8) alkylcarbonyl, nitro, amino, mono- or di-C(1-8) alkylamino, dioxolane, dioxane, dithiolane, and dithione. The C(1-8) alkyl moiety of the substituted C(1-8) alkyl, and the C(1-8) alkyl moeity of the C(1-8) alkoxy, the C(1-8) alkoxycarbonyl, and the mono- and di-lower alkylamino in the substituents of the substituted C(1-8) alkyl group have the same meaning as C(1-8) alkyl defined above. The substituted aryl, the substituted heteroaryl, the substituted aralkyl, and the substituted heteroaralkyl groups each has 1 to 5 independently-selected substituents, such as but not limited to C(1-8) alkyl, hydroxy, C(1-8) alkoxy, carboxy, C(1-8) alkoxycarbonyl, nitro, amino, mono or di-C(1-8) alkylamino, azido, and halogen. The C(1-8) alkyl moiety of the C(1-8) alkyl, the C(1-8) alkoxy, the C(1-8) alkylamino, and the mono- and di-C(1-8) alkylamino groups amoung the susbtituents has the same meaning as C(1-8) alkyl defined above. The heterocyclic group formed with a nitrogen atom includes rings such as, but not limited to, pyrrolyl, piperidinyl, piperidino, morpholinyl, morpholino, thiomorpholino, N-methylpiperazinyl, indolyl, and isoindolyl. The cycloalkyl moeity means a cycloalkyl group of the indicated number of carbon atoms, containing one or more rings anywhere in the structure, such as cycloalkyl groups include cyclopropyl, cyclopropylmethyl, cyclobutyl, cyclopentyl, cyclohexyl, 2-norbornyl, 1-adamantyl and the like. The fluoroalkyl moiety means a lower fluoroalkyl group in which one or more hydrogens of the corresponding C(1-8) alkyl group, as defined above, is replaced by a fluorine atom, such as but not limited to CH₂F, CHF₂, CF₃, CH₂CF₃, and CH₂CH₂CF₃.

The substituents are preferably selected from the group consisting of:
1) H, halogen, nitro, cyano, C(1-8) alkyl, C(1-8) fluoroalkyl, aralkyl, aryl, heteroaryl, C(1-8) alkylcarbonyl, arylcarbonyl, heteroarylcarbonyl, azide, B(OH)₂, and adamantyl;
2) XR¹⁹ wherein X═O or S and R¹⁹ is defined as a C(1-8) alkyl, hydroxyl, C(1-4) alkoxy, fluoroalkyl, aryl, heteroaryl, lower alkylcarbonyl, arylcarbonyl, heteroarylcarbonyl, lower alkylaminocarbonyl, and arylaminocarbonyl; and
3) NR¹⁴R¹⁵ wherein R¹⁴ and R¹⁵ are independently defined as C(1-8) alkyl, or wherein R¹⁴ and R¹⁵ are joined to form an alkyl or heteroalkyl ring system
   wherein said C(1-8) alkyl, C(1-8) fluoroalkyl, aralkyl, aryl, heteroaryl, C(1-8) alkylcarbonyl, arylcarbonyl, heteroarylcarbonyl, and C(1-4) alkoxy may be further substituted, preferably by the substituents 1-3 listed above;

Some of the compounds described herein contain one or more chiral centres and may thus give rise to diastereomers and optical isomers. The present invention is meant to comprehend such possible diastereomers as well as their racemic, resolved and enantiomerically pure forms, and pharmaceutically acceptable salts thereof.

The term “subject” or “patient” as used herein may refer to mammals including humans, primates, horses, cows, pigs, sheep, goats, dogs, cats, rodents, and the like.

The pharmaceutical compositions of the invention are administered to subjects in effective amounts. An effective amount means that amount necessary to delay the onset of, inhibit the progression of, or halt altogether the onset or progression of, or diagnose the particular condition or symptoms of, the particular condition being treated.

An effective amount for treating a neurological disorder is that amount necessary to affect any symptom or indicator of the condition, and/or reverse, halt or stabilize neuronal degradation and/or cell loss that is responsible for the particular condition being treated. In general, an effective amount for treating neuropathies and neuropathic pain will be that amount necessary to favorably affect the neuropathies and/or neuropathic pain. For example, an effective amount for treating neurodegenerative disease of the CNS, such as Alzheimer's disease is an effective amount to prevent memory loss, but is not limited to the amelioration of any one symptom. Similarly, an effective amount for treating Parkinson's disease or ALS is an amount necessary to favorably effect loss of muscular function and/or control, but is not limited to the amelioration of any one symptom. An effective amount for treating glaucoma and macular degeneration is an effective amount to prevent loss of vision. An effective amount for treating a peripheral neuropathy is an effective amount for preventing the development or halting the progression of PNS sensory or motor nerve dysfunction, but is not limited to these symptoms or effects.

In general, an effective amount for treating a mammalian cancer cell proliferation is that amount necessary to affect any symptom or indicator of the condition, and/or reverse, halt or stabilize mammalian cancer cell proliferation and/or migration that is responsible for the particular condition being treated, with that amount being the amount necessary to favorably affect mammalian cancer cell proliferation in vivo.

When administered to a subject, effective amounts will depend, of course, on the particular condition being treated; the severity of the condition; individual patient parameters including age, physical condition, size and weight; concurrent treatment; frequency of treatment; and the mode of administration. These factors are well known to those of ordinary skill in the art and can be addressed with no more than routine experimentation. It is preferred generally that a maximum dose be used, that is, the highest safe dose according to sound medical judgment.

A variety of administration routes are available. The particular mode selected will depend, of course, upon the particular condition being treated, the particular drug selected, the severity of the condition being treated and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, sublingual, topical, nasal, transdermal, intradermal or parenteral routes. The term “parenteral” includes subcutaneous, intravenous (IV), intramuscular, or infusion. Dosage may be adjusted appropriately to achieve desired drug levels, locally or systemically. Generally, daily oral doses of active compounds will be from about 0.01 mg/kg per day to 1000 mg/kg per day. It is expected that intravenous doses in the range of about 1 to 1000 mg/m² per day will be effective. In the event that the response in a subject is insufficient at such doses, even higher doses (or effective higher doses by a different, more localized delivery route) may be employed to the extent that patient tolerance permits.

Compound may be administered as an aqueous and/or non-aqueous solution, being dissolved or suspended in a pharmaceutically acceptable aqueous and/or non-aqueous formulation, prepared by any of the methods well known in the art of pharmacy. These aqueous and/or non-aqueous solutions may contain buffering agents, co-solvents, stabilizers, surfactants, co-solvents and/or encapsulating agents. Buffers and stabilizers are described below, and co-solvents may include HPCD or other encapsulating co-solvents known in the art, PEG and the like.

The solubility of pharmaceutically acceptable salts of 1-a and 1-b can be increased and/or stabilized by the use of an aqueous soluble encapsulating agent. Examples of encapsulating agents include cyclodextrans, such as hydroxypropylcyclodextran (HPCD). Examples of salts include organic and inorganic salts, such as the sodium salt, as well as the salts formed from organic bases, such as ethanolamine, dimethylaminoethanol, and 4-aminopyridine. Use of aqueous 5-45% wt/vol HPCD solutions (either water or saline) are typically preferred for improving the solubility and/or stability of these componds in aqueous media.

The compositions may conveniently be presented in unit dosage form and may be prepared by any of the methods well known in the art of pharmacy. All methods include the step of bringing the conjugates of the invention into association with a carrier that constitutes one or more accessory ingredients. In general, the compositions are prepared by uniformly and intimately bringing the compounds into association with a liquid carrier, a finely divided solid carrier, or both, and then, if necessary, shaping the product.

Compositions suitable for oral administration may be presented as discrete units such as capsules, cachets, tablets, or lozenges, each containing a predetermined amount of the active compound. Other compositions include suspensions in aqueous liquors or non-aqueous liquids such as a syrup, an elixir, or an emulsion.

Other delivery systems can include time-release, delayed release or sustained release delivery systems. Such systems can avoid repeated administrations of the active compounds of the invention, increasing convenience to the subject and the physician. Many types of release delivery systems are available and known to those of ordinary skill in the art. They include polymer based systems such as polylactic and polyglycolic acid, polyanhydrides and polycaprolactone; nonpolymer systems that are lipids including sterols such as cholesterol, cholesterol esters and fatty acids or neutral fats such as mono-, di- and triglycerides; hydrogel release systems; silastic systems; peptide based systems; wax coatings, compressed tablets using conventional binders and excipients, partially fused implants and the like. In addition, a pump-based hardware delivery system can be used, some of which are adapted for implantation.

A long-term sustained release implant also may be used. “Long-term” release, as used herein, means that the implant is constructed and arranged to deliver therapeutic levels of the active ingredient for at least 30 days, and preferably 60 days. Long-term sustained release implants are well known to those of ordinary skill in the art and include some of the release systems described above. Such implants can be particularly useful in treating solid tumors by placing the implant near or directly within the tumor, thereby affecting localized, high-doses of the compounds of the invention.

When administered, the Formulations of the invention are applied in pharmaceutically acceptable compositions. Such preparations may routinely contain salts, buffering agents, preservatives, compatible carriers, and optionally other therapeutic ingredients. When used in medicine the salts should be pharmaceutically acceptable, but non-pharmaceutically acceptable salts may conveniently be used to prepare pharmaceutically acceptable salts thereof and are not excluded from the scope of the invention. Such salts include, but are not limited to, those prepared from the following acids: hydrochloric, hydrobromic, sulphuric, nitric, phosphoric, maleic, acetic, salicylic, p-toluenesulfonic, tartaric, citric, methane sulfonic, formic, malonic, succinic, naphthalene-2-sulfonic, benzene sulfonic, and the like. Also, pharmaceutically acceptable salts can be prepared as alkaline metal or alkaline earth salts, such as sodium, potassium or calcium salts, or from organic bases known in the art such as, but not limited to dimethylaminoethanol, ethanolamine arginine and lysine.

Suitable buffering agents include: phosphate buffers, acetic acid and a salt (1-2% W/V); citric acid and a salt (1-3% W/V); and phosphoric acid and a salt (0.8-2% W/V), as well as others known in the art.

Suitable preservatives include benzalkonium chloride (0.003-0.03% W/V); chlorobutanol (0.3-0.9% W/V); parabens (0.01-0.25% W/V) and thimerosal (0.004-0.02% W/V), as well as others known in the art.

Suitable carriers are pharmaceutically acceptable carriers. The term pharmaceutically acceptable carrier means one or more compatible solid or liquid filler, dilutants or encapsulating substances that are suitable for administration to a human or other animal. The term “carrier” denotes an organic or inorganic ingredient, natural or synthetic, with which the active ingredient is combined to facilitate the application. The components of the pharmaceutical compositions are capable of being commingled with the molecules of the present invention, and with each other, in a manner such that there is no interaction which would substantially impair the desired pharmaceutical efficacy. Carrier Formulations suitable for oral, subcutaneous, intravenous, and intramuscular administration etc., are those which are known in the art.

The compounds of the invention may be delivered with other therapeutic agents. The invention additionally includes co-administration of compound I of the invention with other compounds known to be useful in treating neurodegenerative or proliferative diseases. In neurodegenerative disease this is typified by but not limited to, COX-2 inhibitors, NSAIDS, acetylcholinesterase inhibitors for treating AD, such as tacrine, doneprizil, and rivastigmin, and L-dopa for treating PD, and ACE inhibitors and insulin for the treatment of diabetes. In proliferative diseases such as cancer, this is typified by chemotherapeutics such as Taxol, cisplatin, and the vinca alkaloids.

In the case of peripheral neuropathy induced by a toxic agent, compound I would be delivered separately before, simultaneously with (i.e. independently or in the form of ant-cancer cocktails), or after exposure to the toxic agent. Preferably, compound I and the chemotherapeutic agent are each administered at effective time intervals, during an overlapping period of treatment in order to prevent or restore at least a portion of the neurological function destroyed by the neurotoxic or chemotherapeutic agent. The chemotherapeutic can be any chemotherapeutic agent that causes neurotoxicity, such as dideoxyinosine, deoxy cytizine, D4T, cisplatin, etoposide, vincristine, epithilone or its derivatives, or Taxol™/Taxoter™ and derivatives thereof, which are representative of the classes of agents which induce neuropathies.

By “toxic agent” or “neurotoxic agent” is meant a substance that through its chemical action injures, impairs, or inhibits the activity of a component of the nervous system. Such neurotoxic agents include, but are not limited to, neoplastic agents such as vincristine, vinblastine, cisplatin, Taxol™, D4T or other anti-virals, or dideoxy-compounds, eg., dideoxyinosine; alcohol; metals; industrial toxins involved in occupational or environmental exposure; contaminants in food or medicinals; or over-doses of vitamines or therapeutic drugs, eg. Antibiotics such as penicillin or chloramphenicol, or mega-doses of vitamins A, D, or B6.

In the treatment of cancer where compounds represented by formula I are to be used as pro-apoptotic agents for the killing of cancer cells in vivo compound I would be delivered alone, separately before, simultaneously with (ie. independently or in the form of anti-cancer cocktails), or after treatment with traditional chemotherapeutics such as, but not limited to, Taxol, Taxoter, cisplatin, the vinca alkaloids, and 5-fluorouracil.

### EXAMPLES

Example of compounds represented by formula I are listed below in Table I. Some abbreviations used to indicate substituents are shown below:

TABLE ONE
Examples of Compounds represented by Formula I

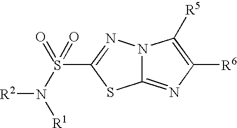

| Abbreviation | Substitution |
|---|---|
| Ph; X = H 4-F—Ph; X = F | 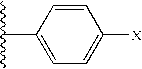 |
| 4-morph-Ph | 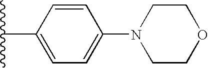 |
| diox-Ph | 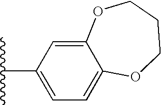 |
| 3′-MeO-biPh; X = OCH₃3′-CF₃-biPh; X = CF₃ | 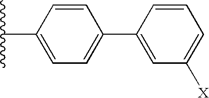 |
| 4-(4-Cl—PhO)Ph | 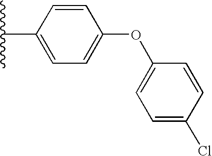 |

| Com- | | | | |
|---|---|---|---|---|
| pound | R¹ | R² | R⁵ | R⁶ |
| 1 | H | H | H | Ph |
| 2 | Na | H | H | Ph |
| 3 | H | H | H | 4-F—Ph |
| 4 | Na | H | H | 4-F—Ph |
| 5 | H | H | H | 4-morph-Ph |
| 6 | Na | H | H | 4-morph-Ph |
| 7 | H | H | H | diox-Ph |
| 8 | Na | H | H | diox-Ph |
| 9 | H | H | H | 3′-MeO-biPh |
| 10 | Na | H | H | 3′-MeO-biPh |
| 11 | H | H | H | 3′-CF₃-biPh |
| 12 | Na | H | H | 3′-CF₃-biPh |
| 13 | H | H | H | 4-(4-Cl—PhO)Ph |
| 14 | Na | H | H | 4-(4-Cl—PhO)Ph |
| 15 | CH₃C(O)— | H | H | Ph |
| 16 | CH₃CH₂CH₂C(O)— | H | H | Ph |
| 17 | tert-BuOC(O)— | H | H | Ph |
| 18 | Boc(H)NCH₂C(O)— | H | H | Ph |
| 19 | TFA.H₂NCH₂C(O)— | H | H | Ph |
| 20 | Ac(H)NCH₂C(O)— | H | H | Ph |
| 21 | 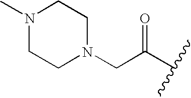 | H | H | Ph |
| 22 | HO₂CCH₂CH₂C(O)— | H | H | Ph |
| 23 | 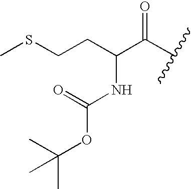 | H | H | Ph |
| 24 | 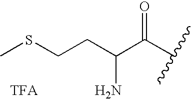 | H | H | Ph |
| 25 | 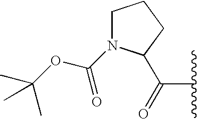 | H | H | Ph |
| 26 | 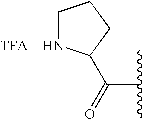 | H | H | Ph |
| 27 | (CH3)2NCH2C(0)- | H | H | 4′-F—Ph |
| 28 | CH₃C(O)— | H | H | diox-Ph |
| 29 | CH₃OCH₂C(O)— | H | H | diox-Ph |
| 30 | CH₃CH₂CH₂C(O)— | H | H | diox-Ph |
| 31 | CH₃C(O)— | H | H | 4-morph-Ph |
| 32 | CH₃OCH₂C(O)— | H | H | 4-morph-Ph |
| 33 | CH₃CH₂CH₂C(O)— | H | H | 4-morph-Ph |
| 34 | CH₃C(O)— | H | H | 3′-MeO-biPh |
| 35 | CH₃OCH₂C(O)— | H | H | 3′-MeO-biPh |
| 36 | CH₃CH₂CH₂C(O)— | H | H | 3′-MeO-biPh |
| 37 | CH₃C(O)— | H | H | 3′-CF₃-biPh |
| 38 | CH₃CH₂CH₂C(O)— | H | H | 3′-CF₃-biPh |
| 39 | CH₃OCH₂C(O)— | H | H | 3′-CF_{3-biPh} |
| 40 | CH₃CH₂CH₂C(O)— | H | H | 3′-CF₃-biPh |
| 41 | 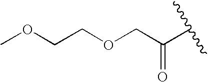 | H | H | 3′-CF₃-biPh |
| 42 | 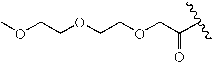 | H | H | 3′-CF₃-biPh |
| 43 | tert-BuOC(O)— | H | H | 3′-CF_{3-biPh} |
| 44 | CH₃C(O)— | H | H | 4-(4-Cl—PhO)Ph |
| 45 | CH₃OCH₂C(O)— | H | H | 4-(4-Cl—PhO)Ph |
| 46 | CH₃CH₂CH₂C(O)— | H | H | 4-(4-Cl—PhO)Ph |
| 47 | 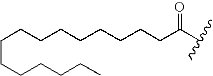 | H | H | 4-(4-Cl—PhO)Ph |
| 48 | PhCH₂OC(O)— | H | H | 4-(4-Cl—PhO)Ph |
| 49 | 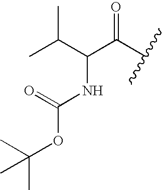 | H | H | 4-(4-Cl—PhO)Ph |
| 50 | 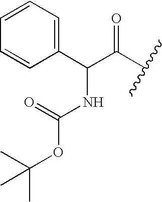 | H | H | 4-(4-Cl—PhO)Ph |
| 51 | 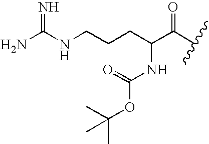 | H | H | 4-(4-Cl—PhO)Ph |

Additional examples of compounds represented by formula I-a are used in Table 2.

TABLE 2
Examples of compounds represented by formula I-a

| Compound | STRUCTURE |
|---|---|
| 52 | 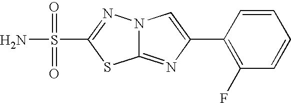 |
| 53 | 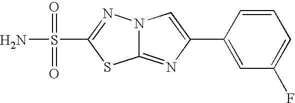 |
| 54 | 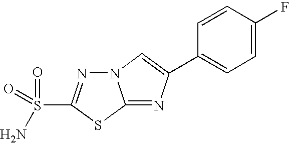 |
| 55 | 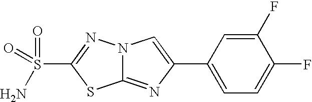 |
| 56 | 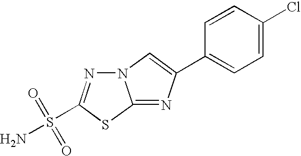 |
| 57 | 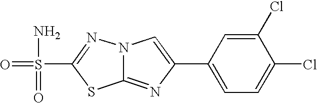 |
| 58 | 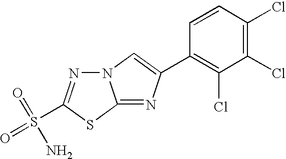 |
| 59 | 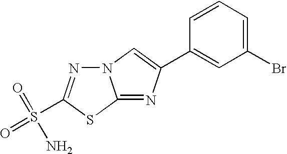 |
| 60 | 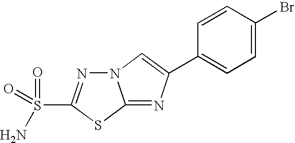 |
| 61 | 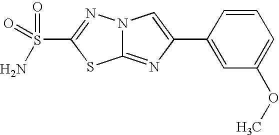 |
| 62 | 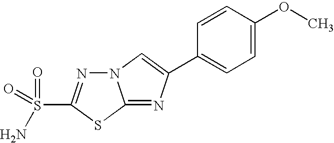 |
| 63 | 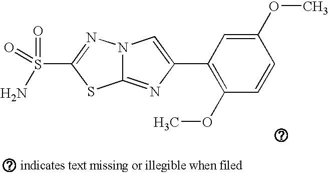 |
| 64 | 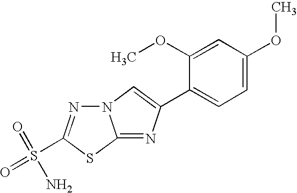 |
| 65 | 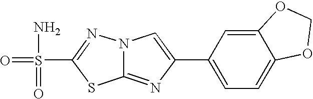 |
| 66 | 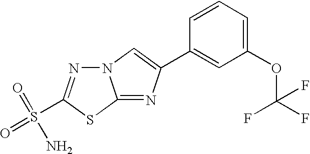 |
| 67 | 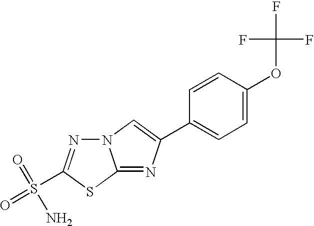 |
| 68 | 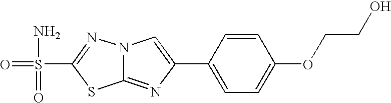 |
| 69 | 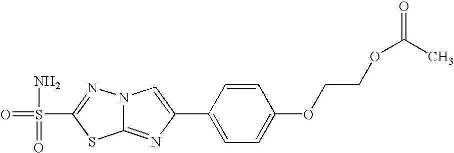 |
| 70 | 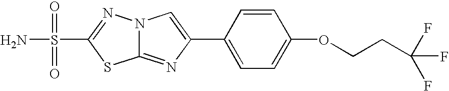 |
| 71 | 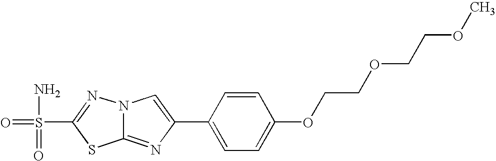 |
| 72 | 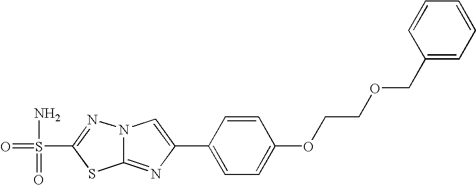 |
| 73 | 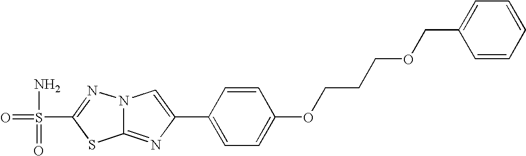 |
| 74 | 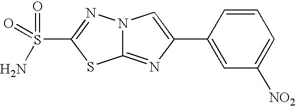 |
| 75 | 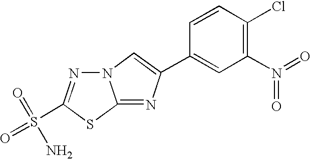 |
| 76 | 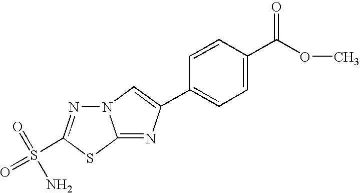 |
| 77 | 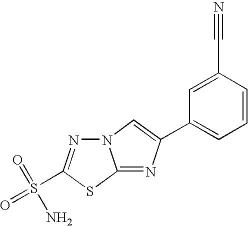 |
| 78 | 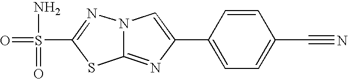 |
| 79 | 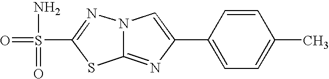 |
| 80 | 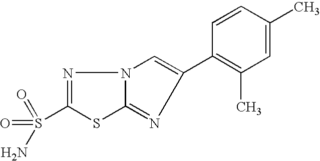 |
| 81 | 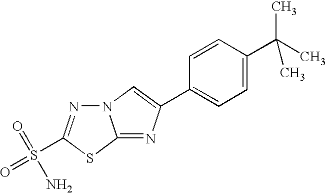 |
| 82 | 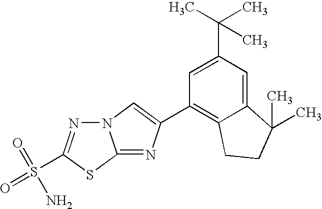 |
| 83 | 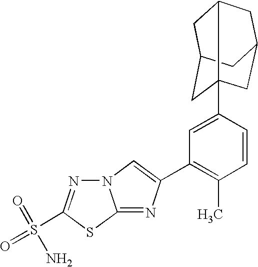 |
| 84 | 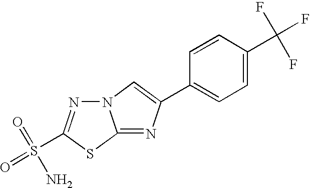 |
| 85 | 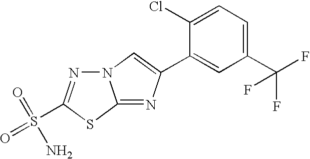 |
| 86 | 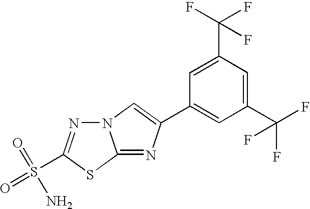 |
| 87 | 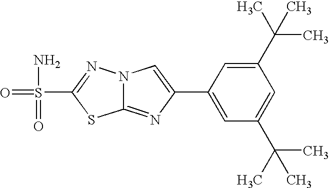 |
| 88 | 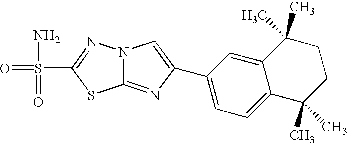 |
| 89 | 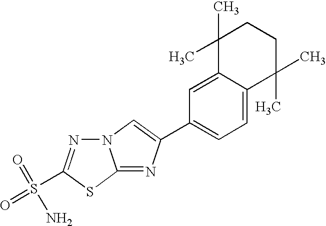 |
| 90 | 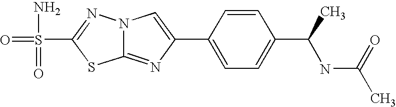 |
| 91 | 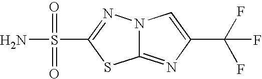 |
| 92 | 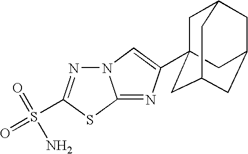 |
| 93 | 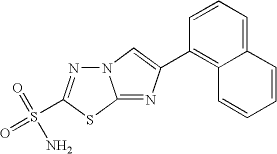 |
| 94 | 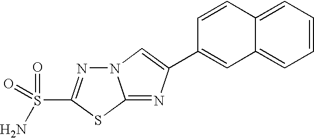 |
| 95 | 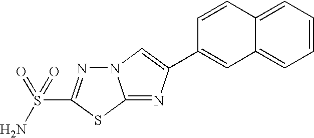 |
| 96 | 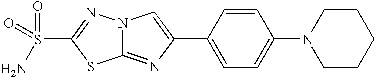 |
| 97 | 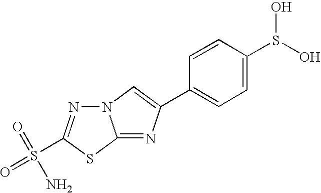 |
| 98 | 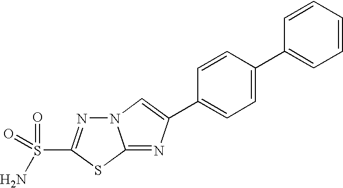 |
| 99 | 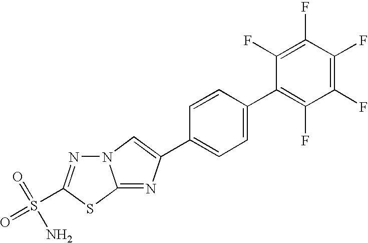 |
| 100 | 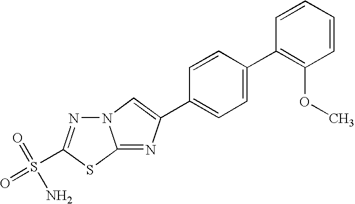 |
| 101 | 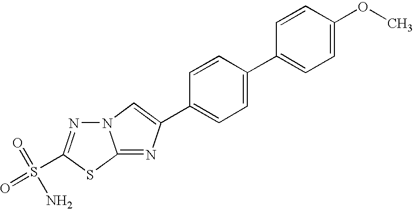 |
| 102 | 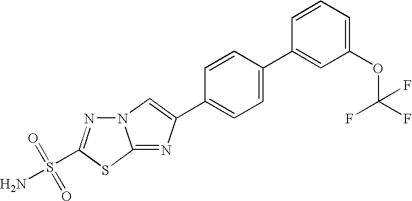 |
| 103 | 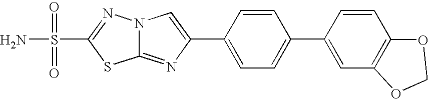 |
| 104 | 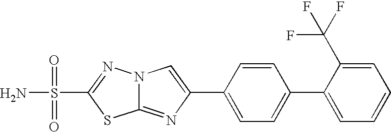 |
| 105 | 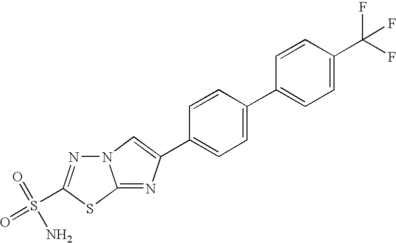 |
| 106 | 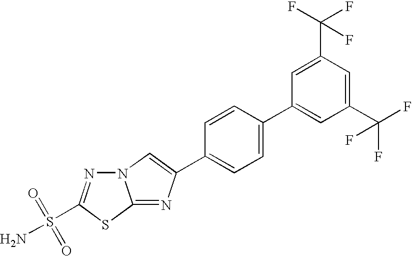 |
| 107 | 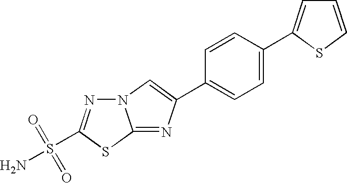 |
| 108 | 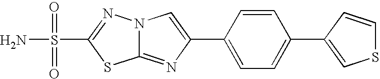 |
| 109 | 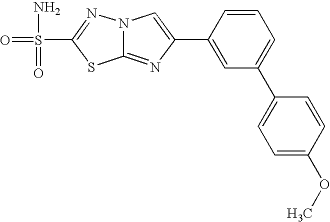 |
| 110 | 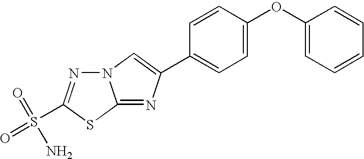 |
| 111 | 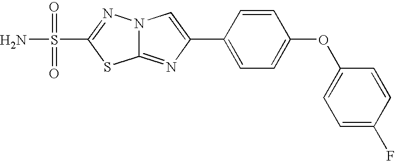 |
| 112 | 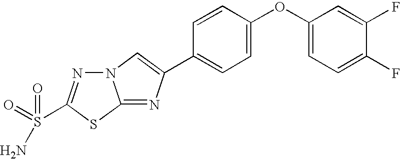 |
| 113 | 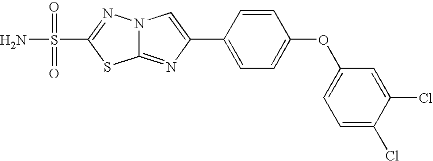 |
| 114 | 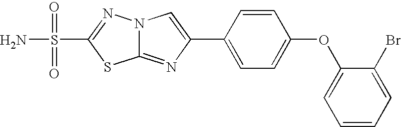 |
| 115 | 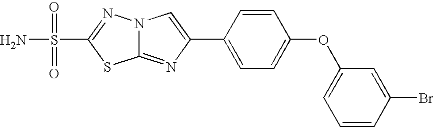 |
| 116 | 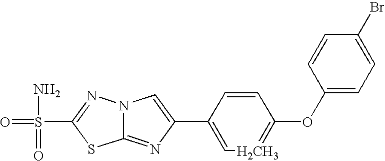 |
| 117 | 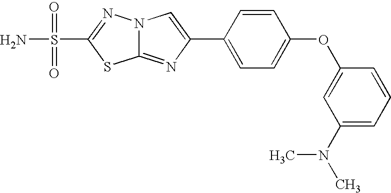 |
| 118 | 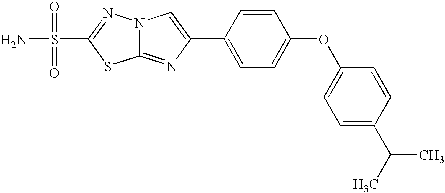 |
| 119 | 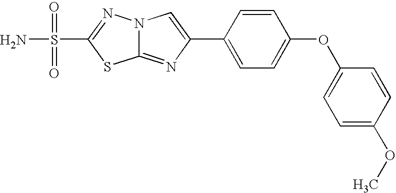 |
| 120 | 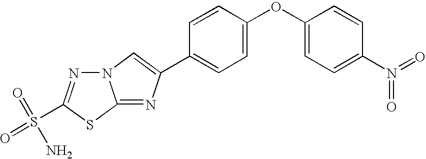 |
| 121 | 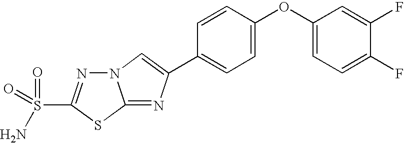 |
| 122 | 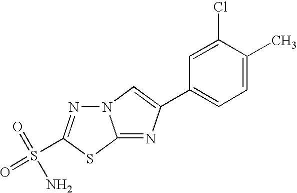 |
| 123 | 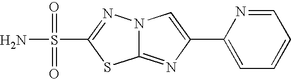 |
| 124 | 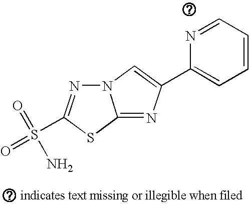 |
| 125 | 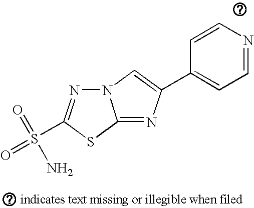 |
| 126 | 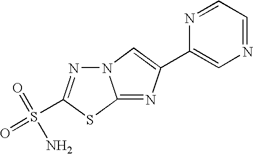 |
| 127 | 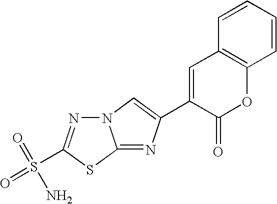 |
| 128 | 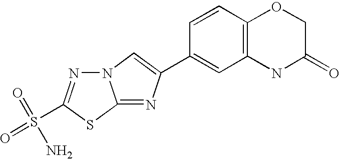 |
| 129 | 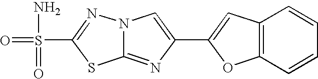 |
| 130 | 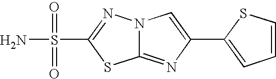 |
| 131 | 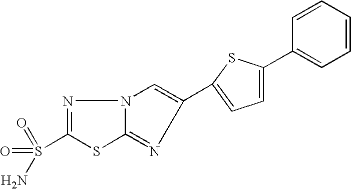 |
| 132 | 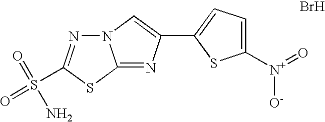 |
| 133 | 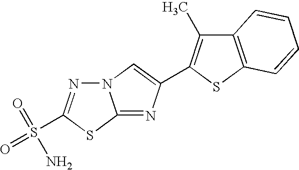 |
| 134 | 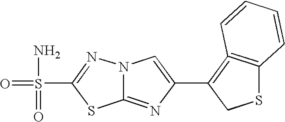 |
| 135 | 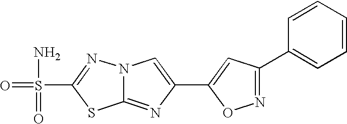 |
| 136 | 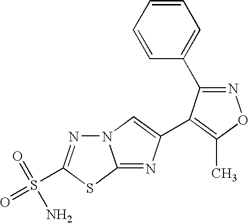 |
| 137 | 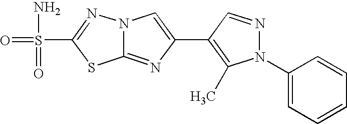 |
| 138 | 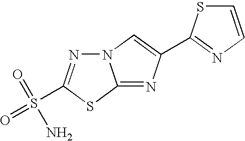 |
| 139 | 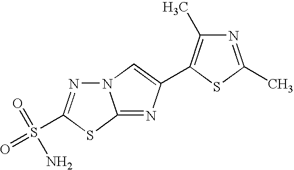 |
| 140 | 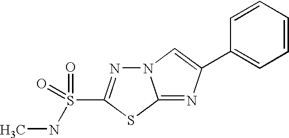 |

A select number of indole and biphenyl derivates, include the following compounds:

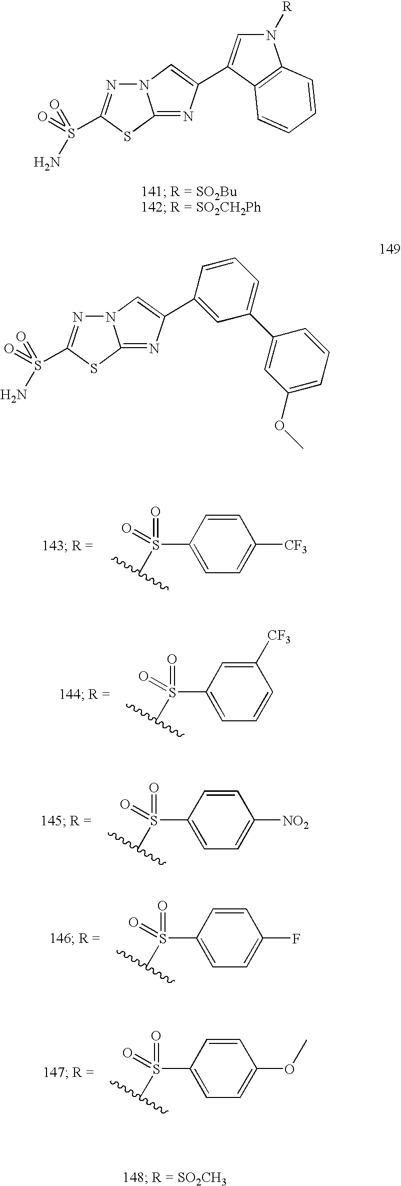

A PEG 400-sebacoylamide derivative of compound 1 is illustrated below: 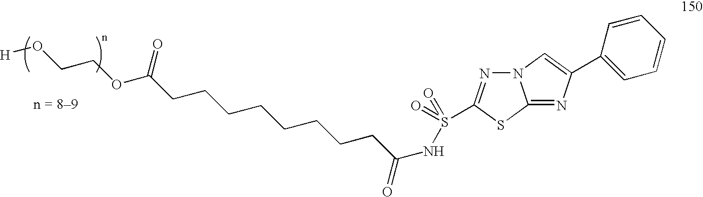
Neuroprotective Effects of Compound Represented by Formula I-a

Several neurotoxic agents and protocols may be used to induce apoptosis in Superior Cervical Ganglion (SCG) neurons. Several of these insults include the withdrawal of trophic support (for example Neuronal Growth Factor (NGF)), treatment with neurotoxic chemotherapeutics such as Taxol™, cisplatin, vincristine, or vinblastine, and treatment with neurotoxic anti-viral agents. Selected compounds represented by Formula I have been found to inhibit apoptosis induced by the above neurotoxic insults.

The Applicant has previously demonstrated that selected compounds represented by Formula I-a (R¹ and R² are selected from H and C(1-4) alkyl protect neurons of the CNS and PNS from various neurotoxic insults (PCT Application No. CA02/01942 (WO 03/051890)). These insults include in vitro treatment of SCG neurons with anti-NGF antibody, Taxol™, cisplatin, and vincristine. Table 3 summarizes a subset of the neuroprotection previously reported.

TABLE 3
Protection of SCG neurons from anit-NGF, Taxol, cisplatin and
vincristine induced cell death

| | anit-NGF | Taxol | | |
|---|---|---|---|---|
| | SCG | SCG | cisplatin SCG | vincristine SCG |
| Compound | IC₅₀ (μM) | IC₅₀ (μM) | IC₅₀ (μM) | IC₅₀ (μM) |
| 1 | 22 | 7 | 5 | 10 |
| 5 | 22 | 7 | | |
| 7 | | 7 | | |
| 9 | | 3 | | |
| 11 | | 2 | | |
| 13 | | 3 | | |
| 143 | | 7 | | |
| 144 | | 10 | | |
| 146 | | 7 | | |
| 147 | | 7 | | |

The above data demonstrates the neuroprotective effect of compounds represented by formula I-a on neurons treated with various neurotoxic agents.

Several neurodegenerative diseases are related to the cellular or functional loss of motor neurons of the CNS and PNS. ALS is a characterized by motor neuron loss as a result of mitochondrial dysfunction, which can be mimicked in culture by the addition of malonate to organotypic brain slices. P1 rat motor cortex brain slices were cultured for 2 weeks prior to drug and malonate addition. After an additional two weeks the slices were fixed and stained with SMI-32 antibody which selectively stains motor neurons found in layer V of the cortex. Compound 13 protected upwards of 80% of these labeled motor neurons at a drug concentration of 1 μM (PCT Application No. CA02/01942 (WO 03/051890)).

Taxol™ commonly causes dose dependent peripheral neuropathies during cancer treatment. When treated with Taxol™ (9 mg/kg in Cremophor EL and ethanol) twice weekly for 3 weeks, Sprague Dawley rats displayed acute symptoms of chemotoxicity, characterized by reduced appetite, weight loss, gait disturbance (a general marker of Taxol™ induced peripheral neuropathy), and general poor health (PCT Application No. CA02/01942 (WO 03/051890)). For example, over a thirteen day period control animals gained an average of 50 g, whereas the Taxol™ treated animals displayed no weight gain. All of the Taxol™ treated animals developed peripheral neuropathies, characterized by ‘tip toe walking’. The extent of this neuropathy was analyzed by quantifying the refracted light captured by a video camera as the animals walked over a glass plate. This data was analyzed by Northern Eclipse software. The Taxol™ treated animals displayed a 46% reduction in foot-pad contact with the glass plate, as compared to control animals. Treatment with compounds 1 (10 mg/kg) resulted in normal weight gain, as compared to control, and a reduction in the severity of the peripheral neuropathies; a 23% loss in foot pad contact was observed, as compared to a 46% loss in the animals treated with Taxol™ alone (PCT Application No. CA02/01942 (WO 03/051890)).

The sciatic nerve crush model is a representative model of axonal repair and regeneration. The sciatic nerve is physically crushed with forceps at the mid-thigh; only the right leg is injured, the left leg serving as a control. The axons die from the crush point to their point of innervation. Functional loss of the axons is rapidly observed as the animals drag their right leg and the toes of the right leg no longer spread. Recovery is observed in approximately 28 days as the animals regain use of their right leg. More quantitative measurements of recovery include toe spread measurements between the digits 1 and 5 and digits 2 and 4, gait analysis and electrical conductivity from the toes to the injury site (PCT Application No. CA02/01942 (WO 03/051890)).

Rats were subjected to the crush injury and treated with either vehicle control or the sodium salts of compounds 1 and 9, compounds 2 and 10 repsectively (1 and 10 mg/kg). Functional recovery was measured as above and improved recovery was observed when the animals were treated with compound. For example, increase toe spread was observed for those animal treated with compound (PCT Application No. CA02/01942 (WO 03/051890)).

Various diseases which result in loss of vision are related to increased inter-ocular pressure and ocular stroke or ischemia. Loss of the dorsal root ganglion (RG) occur during ischemic insult and in diseases such as diabetes and glaucoma. A model of inter-ocular ischemia involves an invasive increase in ocular pressure which results in the collapse of the central retinal artery. Retinal ischemia is confirmed by whitening of the iris and loss of red reflex. The inter-ocular pressure is normalized after 30 minutes. This procedure is performed on the right eye and the left eye serves as a control. Compound 1 was given either by intra-vitrial injection or via SC injections at 10 mg/kg (PCT Application No. CA02/01942 (WO 03/051890)). The health of the RG neurons was assessed by means of histological staining of retinal slices and electro-retinogram (ERG) recordings. Histology of the control animals showed almost complete loss of the RG layer, where as animals treated with compound 1 showed healthy RG layers. Similarly, significant improvements were observed in the ERG for those animals treated with compound verses vehicle control animals. This protection was observed for both the animals which received intra-vitrial injections and those that were treated systemically (SC) (PCT Application No. CA02/01942 (WO 03/051890)).

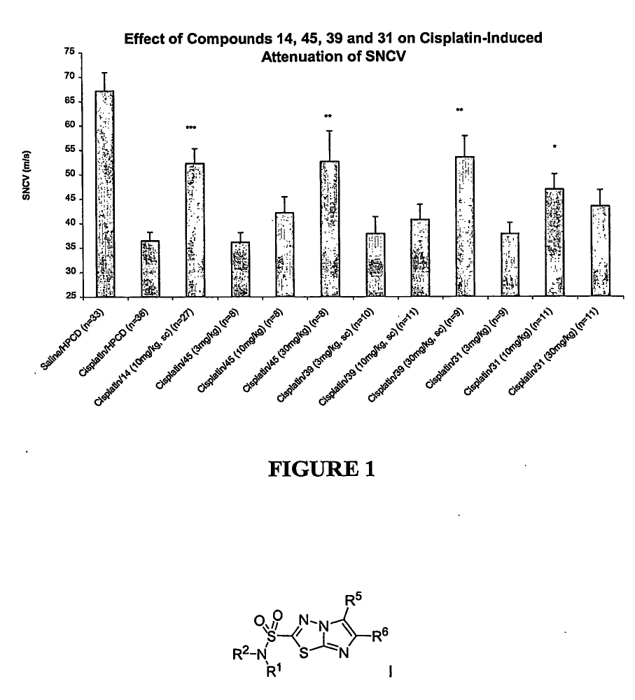

The Applicant herein reports that compounds represented by formula I-a protect rats treated with cisplatin from developing symptoms of peripheral neuropathy. Several primary sulfonamides, represented by formula I-a, such as compounds 2, 6, 10, 12, and 14, display efficacy in this model of peripheral neuropathy. The data and a further discussion is presented later in the text (see Example 151 and FIG. 1).

Improved Formulation of Primary Sulfonamides Represented by Formula I-a

The primary sulfonamides, represented by compounds 1, 3, 5, 7, 9, 11, 13 and 52 through 140 have limited aqueous solubility (<0.5 mg/mL). The sodium salts of compounds 1, 3, 5, 7, 9, and 11, represented by compounds 2, 4, 6, 8, 10, 12, and 14, prepared by the treatment of the parent sulfonamide with 1 equiv of NaOH, display acceptable aqueous solubility (1-10 mg/mL). Use of these sodium salts has allowed for their testing in the above animal models (PCT Application No. CA02/01942 (WO 03/051890)) and various pharmacokinetic studies using percutaneous routes of administration; intravenous (IV), interperantenial (IP), sub-cutaneous (SC), and the like. The modest solubility and long term stability of these solutions can be problematic as the compounds often precipitate with time.

The use of aqueous 5-45% wt/vol HPCD solutions (either water or saline) significantly improves the solubility and/or stability of these Na salts in aqueous media, as displayed below in Table 4.

TABLE 4
Improved solubility of Na salts in aqueous 10 wt/vol % HPCD

| | | Solubility | Stability of |
|---|---|---|---|
| | Solubility | 10 wt/vol % HPCD | HPCD formulation |
| Compound | Water (mg/mL) | water (mg/mL) | (days) |
| 2 | 10 | 20 | >14 |
| 6 | 2.3 | 15 | >14 |
| 10 | — | 10 | >14 |
| 12 | 1.4 | 4 | >14 |
| 14 | 10 | 20 | >14 |

Improved solubility in the presence of HDPC is illustrated, for example, for compound 6. Compound 6 is soluble at 2.3 mg/mL in water. This is significantly increased to >10 mg/mL, with >14 day stability at room temperature, using aqueous 10 wt/vol % HPCD as co-solvent. Similar trends are observed for compounds 2, 10, 12, and 14. These results indicate that the use of HPCD as co-solvent dramatically improves the solubility and stability of aqueous solutions of the sodium salts represented by formula I-a. This is consistant for all o the compounds in Table 4 and is herein extended to compounds 53 to 140.

HPCD formulations of compounds represented by formula I also display improved pharmacokinetic properties as compared to compounds dissolved in water. For example, Compound 1 displays moderate oral bioavailability when administered by gavage at 10 mg/kg, as an aqueous 0.5 wt/vol % CMC/0.5 wt/vol % Tween 80™ suspension (Cₘₐₓ=0.2 μg/mL). A similar semi-suspension of compound 2 (the Na salt of compound 1) provides improved oral bioavailability, however, the inter-animal variation is quite large (Cₘₐₓ=0.56 μg/mL, C_{1/2}=0.9 hrs).

This improved formulation has allowed for administration of compound 2 using percutaneous routes of administration, providing superior plasma drug concentration. When compound 2 is administered at 10 mg/kg SC excellent plasma drug concentrations are observed_(Cₘₐₓ=2.0 μg/mL, C_{1/2}=0.8 hrs). Similarly, compounds 6, 8, 9, 12, 11, and 13 display good pharmacokinetic parameters (plasma Cₘₐₓ=0.8 to 3.0 μg/mL) when administered SC at 10 mg/kg as 10 wt/vol % HPCD solutions.

This improved formulation has allowed for the biological evaluation of a variety of primary sulfonamides, which were previously insoluble or unstable in aqueous media. This formulation also represents a pharmaceutically acceptable formulation in humans at concentrations of 0 to 45% wt/vol HPDC, alone, or in combination with other excipients and surfactants known in the ar of pharmacy.

Compounds listed in Table 2 have been previously disclosed in PCT Application No. CA02/01942 (WO 03/051890). Their respective sodium salts and HPCD formulations thereof are herein included.

Anti-Cancer Activity of the Primary Sulfonamides

Compounds represented by formula I-a display significant pro-apoptotic activity in a number of cancer cell lines including breast, lung, neuroblastoma and medullablastoma cell lines. Select ompounds represented by formula I-a display good microsomal stability (see Table 5) and therapeutic potential.

In order to investigate the anti-cancer potential of compounds represented by formula I-a, 15N neuroblastoma cells lines were treated with compound and assayed for cellular viability after 48 hours. The cellular viability of 15N neuroblastomas treated with compounds 1, 5, 6, 11, and 13 (dissolved in DMSO) are summarized in Table 5 (see Compound 152).

TABLE 5
Anti-cancer activity and microsomal stability of compounds 1, 5, 9, 11, 13
and 5-Br-6-Ph-ITS.

| Compound | 15N | Microsomal |
|---|---|---|
| (Na salt) | IC₅₀ (μM) | Stability (1 hr)¹ |
| 1 (2) | 20 | 90% |
| 5 (6) | 20 | 100% |
| 9 (10) | 5 | 74% |
| 11 (12) | 2 | 60% |
| 13 (14) | 10 | 85% |
| 5-Br-6-Ph-ITS | 3 | 0% |

A significant structure activity relationship (SAR) is observed. Compounds 1 and 5 displayed mild anti-cancer effect with IC₅₀s of approximately 20 μM. An increase in hydrophobic substitution at R⁶ leads to a 3-10 fold increase in pro-apoptotic activity. Compounds 9 and 13 display IC₅₀s of 5 and 10 μM, respectively. Compound 11 demonstrates a 10 fold increase in activity over the parent compound, compound 1, with an IC₅₀ of 2 μM.

A significant correlation is made between the neuroprotective and anti-cancer activity of compounds represented by formula I-a. Those compounds which are more potent neuroprotective agents, for example compounds 9, 11, and 13, are also more potent anti-cancer agents, and vise versa.

Previous reports have demonstrated that 5-Bromo-6-phenylimidazo[2,1-β]-1,3,4,-thiadiazole-2-sulfonamides (R¹═R²═H, R⁵═Br, R³═Ph; 5-Br-6-Ph-ITS) displays anti-proliferative activity (Gadad, A. K. India. Arzneim.-Forsch., 49(10), 858-863, 1999). The Applicant herein demonstrates that compound 11 is more potent than 5-Br-6-Ph-ITS. Compounds represented by formula I-a display pharamecuetically acceptable microsomal stability. In contrast, 5-Br-6-Ph-ITS is rapidly consumer by microsomal fractions suggesting limited clinical potential for this compound. The use of select compounds represented by formula I-a, therefore, represent a novel approach to the treatment of various cancers such as, but not limited to, neuroblastoma.

The above assays demonstrate the pro-apoptotic potential of these compounds; however, dying cells may still stain positive, underestimating the overall potency of the compound. The Applicant has developed cloneogenic assays for these and other cell lines in order to further demonstrate the anti-cancer potency of compounds represented by formula I-a. In this paradigm, Du145 prostate, HCT116 colon, 15N Neuroblastoma, IMR32 Neuroblastoma, Daoy Medulloblastoma, and MDAMB231 breast cells are individually plated and allowed to proliferate for 48 hours. Compound is added to the culture and left on for 24 hours, at which time both compound and dead cells are washed off the plate. Fresh media is added and the cells are allowed to grow for an additional 7-10 days. The remaining healthy cells reproduce and formed localized colonies. These colonies are counted and EC₅₀ values are determined relative to non-treated controls. The results are summarized in Table 6 (see Compound 153).

TABLE 6
Clonogenic assays with compounds 1, 11, and 13.

| | ED₅₀ of | ED₅₀ of | ED₅₀ of |
|---|---|---|---|
| | Compound | Compound | Compound |
| Cell line | 1 (μM) | 11 (μM) | 13 (μM) |
| Du145 (prostate) | 8 | | 8 |
| HCT116 (colon) | 12 | 1.5 | |
| 15 N (neuroblastoma) | 7 | 0.75 | |
| IMR32 (neuroblastoma) | | | 0.75 |
| Daoy (medulloblastoma) | >5 | 1.0 | |
| MDAMB231 (breast) | >5 | 1.0 | |

When compounds 1, 11, and 13 were tested in this paradigm a similar trend was observed for potency ranking of compounds 1, 11, and 13. Compound 1 displays IC₅₀s in the range of 5 to 12 μM. In general compounds 11 and 13 are more potent that compound 1 with IC₅₀s ranging from 0.75 to 8 μM; usually no colonies are observed at concentration greater than 1 to 5 μM. These results demonstrate the significant anti-cancer potency of select compounds represented by formula I-a, against a wide range of cancer cell types.

Although 5-Br-6-Ph-ITS displays a significant activity, as compared to compound 1, it is accompanied by a dramatic loss in microsomal stability. The more hydrophobic derivatives of compound 1, such as compound 9, 11, and 13 display similar or better cellular activity to 5-Br-6-Ph-ITS. These latter compounds display low micromolar, pro-apoptotic activity towards cancer cells, stability, solubility as their sodium salts, and pharmacokinetics, representing pharmaceutically viable compounds for the treatment of a wide range of different cancer types such as, but not limited to, prostate, colon, neuroblastoma, medulloblastoma, and breast cancer. These cancers vary greatly in their place of origin, tumor morphology, proliferation rate, and potential for metastases, suggesting that compounds represented by formula I-a are useful in the treatment of a wide range of cancer types.

Compounds listed in Table 2 have been previously disclosed in PCT Application No. CA02/01942 (WO 03/051890). Their respective sodium salts, and those of compounds represented by compounds 141 to 149, and HPCD formulations thereof are herein included for the treatment of cancer.

N-Acyl Sulfonamides

Compounds represented by formula I-a display both neuroprotective and anti-cancer activity. These compounds display limited aqueous solubility (<1 mg/mL), however, their sodium salts and HPCD formulations thereof display aqueous solubility and stability in the range of 5-25 mg/mL. Solutions formulated using HPCD as a co-solvent display improved solubility and stability. The pH of said formulations are generally in the range of 7.6-9.2. A pharmaceutically acceptable pH range is approximately 4.5 to 8.6.

The N-acylsulfonamides represented by formula I-b may be formulated at near neutral pH (7.4). Compounds represented by formula I-b display good pharmacokinetic (PK) profiles and are de-acylated (cleaved) in vivo to the primary sulfonamides represented by formula I-a. The PK profile of the free primary sulfonamide are similar to that of the Na-salts of the primary sulfonamide, delivered at the same dose. In this way, the N-acyl sulfonamides represented by formula I-b act as prodrugs for the delivery of the primary sulfonamides represented by formula I-a.

The use of simple N-acetyl, N-propionyl, and N-butanoylsulfonamides as prodrugs has been previously described for the COX-2 inhibitors parecoxib sodiumand celecoxib (Talley, J. J., et. al., J. Med. Chem. 2000 May 4; 43(9):1661-3 and Mamidi, R. N., et al. Biopharm. Drug Dispos. 2002 October; 23(7):273-82). As stated above, select N-acylsulfonamides are converted in vivo to the corresponding primary sulfonamide and a carboxylic acid. N-Acylsulfonamides display altered solubility and pharmacokinetic parameters as compared to the corresponding primary sulfonamide.

The synthesis and biological evaluation of a wide range of N-acylsulfonamide derivatives, represented by formula I-b, of the primary sulfonamides represented by formula I-a is disclosed. Select compounds are summarized in Table I as compounds 15 through 51. A range of N-acetyl functionalities were incorporated in order to control gastric and/or cellular absorption. N-acetyl chain length was investigated in terms of aqueous solubility, lipophilicity and rate of metabolism to the primary sulfonamide. The N-acyl moieties range in length from acetyl (C2) to palmatoyl (C16). The complexity of the N-acetyl groups range from amino acid derivatives to polyethers. The utility of each of these groups differ significantly. Short chain N-acyl groups or polar/basic functionalities are intended to facilitate aqueous solubility for oral and/or percutaneous routes of administration. Medium to long chain N-acyl groups are intended to facilitate lipid solubility in oral and/or trans-dermal/topical routes of administration. Various di- and tri-amino acid receptors are known to facilitate active transport of compounds in cell types such as gastric and cancer cell lines. Thus, variation of the N-acyl moiety will effect the delivery, pharmacokinetics, and conversion rates of compounds represented by formula I-b.

Deprotonation of select N-acetylsulfonamides represented by formula I-b, the free acid, with 1 equiv of NaOH yields the corresponding sodium salt. Alternatively, the sodium salt can be prepared in situ by dissolving the free acid in phosphate buffered saline (PBS) that has been buffered to a pH of 7.4. The solubility and stability of these solutions can be improved by the use of an aqueous soluble co-solvent such as, but not limited to HPCD. This solubility can be further improved by the addition of surfactants such as PEG 400. In general, the free acid is suspended in 10 wt/vol % HPDC (10 g dissolved in 100 mL water) and treated with 0.5M PBS (pH 7.4) such that the volumes are in a ratio of 75:25. Vortexing and/or sonication for 1-10 minutes provides a clear solution (filtration of particulate matter may be required). This is illustrated for compound 15 in Table 7.

TABLE 7
Formulation of Compound 15

| | Solubility | | |
|---|---|---|---|
| Formulation | (mg/mL) | Stability | |
| 1000 μL 10% HPCD or 20% PEG 400 | 0 | — | |
| 1000 μL 0.5 M PBS | 2.5 | 1-2 | days |
| 250 μL 0.5 M PBS | 10 | >4 | weeks |
| 750 μL 10% HPCD | | | |
| 250 μL 0.5 M PBS | 25 | >4 | weeks |
| 250 μL 10% HPCD | | | |
| 500 μL 20% PEG 400 | | | |

10 wt/vol HPCD - 10 g of HPDC dissolved in 100 mL of water.
20 vol/vol % PEG 400 - 2 mL PEG 400 dissolved in 8 mL of water.

Compound 15 is not directly soluble in aqueous HPDC or PEG 400 but is mildly soluble in 0.5M PBS (pH 7.4). The combination of PBS and HPCD (25:75) increases this solubility to 10 mg/mL. This solution is stable for greater than 4 weeks. A 2 fold increase in solubility may be obtained by using a combination of aqueous PBS, HPDC and PEG 400, as described above. The aqueous solubility of Na-15 is dramatically increased when the encapsulating agent HPDC is used, and this solubility may be further augmented by the use of other excipients such as PEG 400.

The general protocol of PBS/HPCD (25:75) was useful for the dissolution of select compounds represented by formula I-b is summarized in Table 8.

TABLE 8
Solubility of compounds of formula l − b in PBS/HPCD solution

| | Solubility in | Stability in | | | |
|---|---|---|---|---|---|
| Compound | 10 wt/vol HPCD | 10 wt/vol HPCD | | log P | pH |
| 15 | 10 | >4 | weeks | 2.6 | 7.4 |
| 16 | 4 | >4 | week | 3.67 | 7.4 |
| 18 | 10 | >4 | weeks | 3.32 | 7.4 |
| 20 | 10 | >4 | weeks | 1.45 | 7.4 |
| 22 | 8 | >4 | weeks | 2.26 | 7.4 |
| 23 | 5 | >1 | week | 3.37 | 7.4 |
| 31 | 10 | >4 | weeks | 2.48 | 7.4 |
| 32 | 0 | — | | 2.14 | — |
| 37 | 10 | >1 | week | 5.19 | 7.4 |
| 38 | 0 | — | | 5.58 | — |
| 39 | 10 | >1 | week | 4.85 | 7.4 |
| 40 | 5 | >1 | week | 6.26 | 7.4 |
| 41 | 10 | >1 | week | 4.96 | 7.4 |
| 42 | 10 | >4 | weeks | 4.35 | 7.4 |
| 43 | 5 | >1 | week | 5.76 | 7.4 |

In general, the above N-acetyl derivatives are soluble at 10 mg/mL, while N-butanoyl derivatives are less soluble at 4-5 mg/mL. This drop in solubility appears to related to the N-acetyl group and does not correlate well with the log P of the compounds. Compounds 32 and 38 are not soluble using this formulation. The reason for this lack of solubility is unclear as it does not correlate with the log P of the compounds, but may be due to poor interactions of the propionyl group with the HPCD.

The solubility of compound 38 was further investigated by incorporating PEG 400 into the formulation. These results are summarized below.

| Formulation | Solubility (mg/mL) |
|---|---|
| 250 μL 0.5 M PBS | 0 |
| 750 μL 10% HPCD | |
| 250 μL 0.5 M PBS | 4 |
| 250 μL 10% HPCD | |
| 500 μL 20% PEG 400 | |
| 250 μL 0.5 M PBS | 0 |
| 750 μL 20% PEG 400 | |
| 50% PEG 400/ethanol | 10 |

Compound 38 is not soluble in binary PBS/HPCD or PBS/PEG 400 formulations, however, the combination of PBS, 10% HPCD, and 20% PEG 400 provides a solution at 4 mg/mL, which is stable for more than 4 weeks. Compound 38 is also soluble at 10 mg/mL in a non-aqueous formulation composed of 50:50 PEG 400 and ethanol.

Compound 31. MeSO₃H is not soluble in 10% HPCD, however, it is completely soluble in dimethylacetamide (DMAc), which may be diluted with water to 25:75 DMAc/water, to provide a 5 mg/mL solution with a pH of 5.4.

The TFA salts 24 and 26 are not soluble in water or in 10% HPCD, however, once neutralized using the PBS/10 wt/vol % HPCD (25:75) there are soluble at 4-5 mg/mL. These compounds also represent starting materials for further elaboration of the N-acyl poly-amino acid side chains.

Pharmaceutically acceptable organic bases such as, but not limited to, ethanolamine, dimethylaminoethanol and 4-aminopyridine, may be used to deprotonate the N-acylsulfonamide, and provide aqueous soluble formulations. In this way, addition of 1 equiv of ethanolamine, dimethylaminoethanol or 4-aminopyridine to a suspension of compound 15 in 10 wt/vol % HPCD will yield a clear solution at 5-10 mg/mL.

Similarly, the addition of ethanolamine (20 μL) to a suspension of 25 mg of compound 15 or 37 suspended in 1 mL PEG 400/ethanol (50:50) provides a clear solution, which may be further diluted up to 5 fold with water, without precipitation.

Compound 150 is freely soluble in alcohols such as ethanol and may be dissolved at 10-20 mg/mL in the formulation described above (250 μL PBS, 250 μL 10% HPCD, and 500 μL 20% PEG 400).

Therefore, the disclosed compounds represented by formula I-b, and/or their organic or inorganic salts, display good solubility in aqueous and non-aqueous media, finding use in various routes of administration well known to those in the art of pharmacy.

Compound 15 is converted to compound 1 in the presence of liver microsomes using the procedure described by Cresteil, T., et al. (Cresteil, T., et al. Am. Soc. Pharm. Exper. Therapeutics, 2002, 30, 438-445). Upwards of 50% conversion is observed after 60 minutes. When incubated with rat primary hepatocytes, the conversion of compounds 15, 37, and 44 to their respective primary sulfonamides, 1, 11, and 13, is observed. After being incubated from 90 minutes conversion rates of 6, 18, and 12% were observed for compounds 15, 27, and 44, respectively.

When administered to rats subcutaneously, compound 15 (10 mg/mL) is well distributed (Cₘₐₓ=20 μg/mL in plasma). Conversion of compound 15 to 1 is observed with plasma levels of 1 reaching a Cₘₐₓ of 0.5-1 μg/mL. Conversion of compound 16 to compound 1 is observed with plasma levels of 1 reaching a Cₘₐₓ of 3 μg/mL Similar in vivo conversion is observed for select compounds listed in table 5 with whole blood drug concentrations being similar to that of their respective primary sulfonamide sodium salts, represented by formula I-a, administered at the same dose in aqueous 10 wt/vol % HPCD.

When treated with chemotherapeutic agents such as Taxol and cisplatin rats develop various symptoms of peripheral neuropathy. Compounds represented by formula I-a and I-b prevent a cisplatin mediated reduction in sensor nerve conduction velocity (SNCV).

Male Sprague-Dawley rats were administered 2.5 mg/kg cisplatin daily, for five consecutive days to achieve a final cumulative dose of 12.5 mg/kg. On the third day following the final cisplatin injection, animals received compounds SC at concentrations of (3, 10, and 30 mg/kg). Dosing continued Monday through Friday for three consecutive weeks. The effect of cisplatin on peripheral nerve function, and the ability of the compounds to attenuate the cisplatin effect were determined after three weeks of drug treatment by measuring the sensory nerve conduction velocity (SNCV) in the caudal nerve of the tail. Stimulating electrodes were used to deliver 2 mA pulses once per second for 1.5 min. The resulting compound sensory nerve action potentials were averaged, and the mean response onset time was determined from the averaged response. Two mean response times were determined, the second being 20 mm distal from the first. The difference in onset time between the two recordings was determined and used to calculate the conductance velocity.

In general, rats treated with cisplatin display a reduced maturational increase in SNCV as compared to control animals. This loss in SNCV is prevented by treatment with compound 14 (10 and 30 mg/kg). Similarly, compounds 2, 6, 10, 12 are protective at 10-30 mg/mg (data not shown). The N-acyl derivatives of these compounds also demonstrate protective activity in this model 45, 39, and 31 (30 mg/kg), demonstrating that the N-acyl prodrugs are converted and active in an in vivo model of peripheral neuropathy. Therefore, compounds represented by formula I-b are useful in the treatment of neurodegenerative diseases such as, but not limited to, peripheral neuropathies (see Compound 151 and FIG. 1).

Taken together, compounds represented by formula I-b are novel aqueous soluble prodrugs of the primary sulfonamides represented by formula I-a. These prodrugs may be cleaved in in vitro and in vivo to yield the desired primary sulfonamides. The primary sulfonamides represented by formula I-a display therapeutic potential in the treatment of neurodegenerative diseases (as exemplified in PCT Application No. CA02/01942 (WO 03/051890)) and in the treatment of proliferative disorders such as cancer, as disclosed herein. The novel compounds represented by formula I-b are effective prodrugs of compounds represented by formula I-a. These compounds display aqueous solubility at near neutral pH, representing an alternative delivery system for the primary sulfonamides. Compounds represented by formula I are useful in the treatment of neurodegenerative diseases and proliferative disease such as cancer.

Synthetic Procedures

Compounds of the present invention may be prepared in the following manner.

Imidazo[2,1-b]-1,3,4,-thiadiazole-2-sulfonamides may be prepared by the condensation of 2-amino-1,3,4-thiadiazole-5-sulfonamide with various α-bromoacetophenones using known procedures (see PCT Application No. CA02/01942 (WO 03/051890) and references therein).

Acylation of the primary sulfonamide with the appropriate acyl anhydride or acyl chloride in a solvent such as THF yields the desired N-acyl sulfonamides.

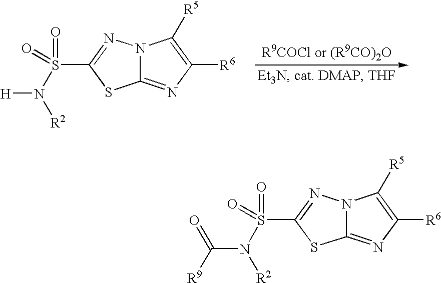

Coupling of the sulfonamide with an appropriately protected α-amino acid or peptide fragment using 2-chloro-1-methylpyridinium iodide provides the desired N-(2-protected-amino)acyl sulfonamide derivatives, as illustrated below.

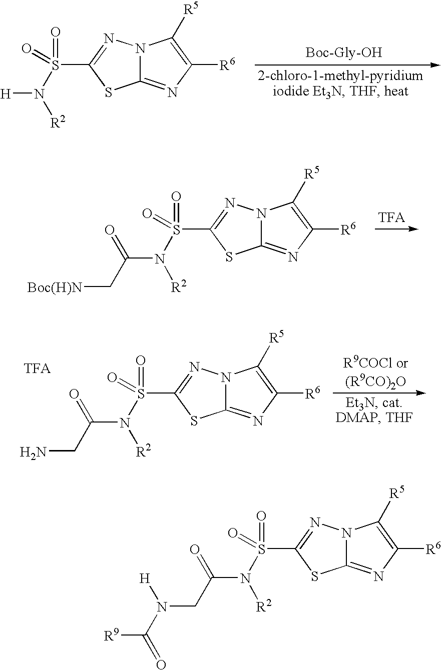

Deprotection using an appropriate reagent, in this case the Boc group is removed using an acid such as TFA to provide the TFA salt. The resulting N-(2-amino)acyl sulfonamide may be further modified by method known in the art; in this case acylation with an appropriate acyl chloride. This coupling reaction works well with various activated amino acids such as succinate and pentaflourophenyl esters, however, DIC/HOBt couplings provide lower yields. The method described herein extends to all other coupling protocols known in the art which provide the desired N-acyl sulfonamide and the use or various protecting group protocols known in the art.

Coupling of the primary sulfonamides with various carboxylic acids works well using 2-chloro-1-methylpyridinium iodide as the coupling agent.

Compounds 1, 3, 5, 7, 9, 11, and 13 were prepared as previously described (see PCT Application No. CA02/01942 (WO 03/051890) and references therein).

General Procedure for the Preparation of Na Salts Represented by Formula I-a.

Compounds 2, 4, 6, 8, 10, 12, and 14 were prepared by independently suspending compounds 1, 3, 5, 7, 9, 11, and 13, respectively, in a 3:2:1 THF/EtOH/water solution and adding 1 equiv of NaOH dissolved in a minimum of water. After 30 minutes volatiles were removed under reduced pressure to provide the desired sodium salts, as previously described (PCT Application No. CA02/01942 (WO 03/051890)).

Compound 15.

Compound 1 (500 mg, 1.8 mmol) was dissolved in THF (10 mL) and treated with triethylamine (532 μL, 3.90 mmol) and acetic chloride (140 mL, 1.96 mmol). The solution Was stirred for 16 hr before 1M HCl was added (20 mL). The resulting solid was filtered and triturated with MeOH (3×5 mL) to provide compound 15 as a white solid (95% yield). ¹H NMR (200 MHz, DMSO-d₆) δ 8.67 (s, 1H), 7.89 (d, 2H), 7.43 (t, 2H), 7.36 (t, 1H), 2.00 (s, 3H). MS (API-ES, positive scan, m/z) M+1=323.1

Compound 16:

Compound 16 was prepared as per compound 15, using butyric anhydride, to provide a white solid after triturating with MeOH. ¹H NMR (200 MHz, DMSO-d⁶) δ 8.87 (s, 1H), 7.89 (d, 2H), 7.43 (t, 2H), 7.36 (t, 1H), 3.38 (q, J=7.8 Hz, 2H), 1.43 (sept, J=7.8z, 2H), 1.06 (t, J=7.8 Hz, 3H).

Compound 17:

Compound 15 (2.20 g, 7.92 mmol) was suspended in THF (120 mL) and treated with Boc₂O (2.03 g, 9.3 mmol) and triethylamine (1.10 mL, 7.9 mmol). The solution was stirred for 36 hours. Saturated aqueous NH₄Cl (5 mL) and ethyl acetate (20 mL) were added and the organic layer was washer with brine (2×10 mL), dried over anhydrous MgSO₄, filtered and the solvent removed under reduced pressure. The resulting solid was puriied by silica gel chromatography, eluting with 40:60 THF/hexane, to provide an oil which was dried overnight under high vacuum to provide compound 17 as a white solid (3.00 g). ¹H NMR (200 MHz, DMSO-d⁶) δ 8.71 (s, 1H), 7.87 (d, J=8.3 Hz, 2H), 7.40 (m, 2H), 7.29 (m, 1H), 1.24 (s, 9H).

Compound 18:

Compound 15 (3.60 g, 10.0 mmol) was suspended in THF (5 mL) and treated with Boc-Gly-OSu (1.60 g, 16.0 mmol) and triethylamine (3.0 mL, 22.0 mmol). The solution was stirred for 36 hours. Saturated aqueous NH₄Cl (5 mL) and ethyl acetate (20 mL) were added and the organic layer was washer with brine (2×10 mL), dried over anhydrous MgSO₄, filtered and the solvent removed under reduced pressure. The resulting solid was crystallized from cold ethyl acetate to provide an off white solid (1.80 g, 41%). ¹H NMR (200 MHz, DMSO-d⁶) δ 8.67 (s, 1H), 1.86 (s, J=7.3 Hz, 2H), 7.40 (t, J=7.3 Hz, 2H), 7.30 (t, J=7.3 Hz, 1H), 6.47 (br t, 1H), 3.45 (br d, 2H), 1.33 (s, 3H).

Compound 19:

Compound 19 (0.39 g) was suspended in trifluoroacetic acid (3 mL) and 3 drops of water were added. The solution was stirred for 30 minutes and volatiles were removed under reduced pressure to provide compound 19 in quantitative yield. ¹H NMR (200 MHz, DMSO-d⁶) δ 8.70 (s, 1H), 7.86 (d, J=7.0 Hz, 2H), 7.79 (br s, 1H), 7.40 (t, J=7.0 Hz, 2H), 7.28 (t, J=7.0 Hz, 1H), 3.44 (m, 2H).

Compound 22:

Compound 15 (360 mg, 1.0 mmol) was suspended in THF (5 mL) and treated with succinic anhydride (160 mg, 1.6 mmol) and triethylamine (306 μL, 2.2 mmol). The solution was stirred overnight. Saturated aqueous NH₄Cl (5 mL) and ethyl acetate (20 mL) were added and the organic layer was washer with brine (2×10 mL), dried over anhydrous MgSO₄, filtered and the solvent removed under reduced pressure. The resulting solid was triturated with MeOH (10 mL) to provide an off white solid (192 mg). ¹H NMR (200 MHz, DMSO-d⁶) δ 8.87 (s, 1H), 7.88 (d, J=7.4 Hz, 2H), 7.42 (t, J=7.4 Hz, 2H), 7.31 (t, J=7.3 Hz, 1H), 2.54-2.35 (m, 4H).

Compound 23:

Compound 23 was prepared as described for compound 18 using Boc-Met-OSu instead of Boc-Gly-OSu. The crude reaction mixture was purified by silica gel chromatography, eluting with a linear gradient of 0-75% MeOH/CH₂Cl₂, to provide compound 23 as a white solid (280 mg). ¹H NMR (200 MHz, DMSO-d⁶) δ8.16 (s, 1H), 7.72 (d, J=7.3 Hz, 2H), 7.40-7.20 (m, 3H), 5.61 (br s, 1H), 4.23 (m, 1H), 2.63 (m, 2H), 1.92 (s, 3H), 1.95-1.90 (m, 2H), 1.32 (s, 9H).

Compound 24:

Compound 26 was suspended in trifluoroacetic acid (3 mL) and 3 drops of water were added. The solution was stirred for 30 minutes and volatiles were removed under reduced pressure to provide compound 24 in quantitative yield. ¹H NMR (200 MHz, DMSO-d ⁶) δ 8.73 (s, 1H), 7.94 (br s, 2H), 7.91 (d, J=7.9 Hz, 2H), 7.38 (t, J=7.1 Hz, 2H), 7.27 (t, J=7.2 Hz, 1H), 3.67 (br d, 1H), 2.60 (s, 3H), 2.58 (m, 2H), 2.04 (m, 2H). LCMS M+1=412.1.

Compound 25:

Compound 25 was prepared as described for compound 18 using Boc-Pro-OSu instead of Boc-Gly-OSu, to provide a 1.5:1 inseparable (silica gel or C18 chromatography) mixture to compounds 25 and 1. This crude mixture was advanced to the next step without further purification (Compound 25).

Compound 26:

The semi-crude reaction mixture from Compound 25 was suspended in trifluoroacetic acid (5 mL) and 3 drops of water were added. The solution was stirred for 30 minutes and volatiles were removed under reduced pressure. The resulting solid was triturated with hot ethyl acetate (10 mL) to provide compound 26 as an off white solid (210 mg). ¹H NMR (200 MHz, DMSO-d⁶) δ 9.01 (br s, 1H), 8.72 (s, 1H), 8.30 (br s, 1H), 7.86 (d, J=7.3 Hz, 2H), 7.37 (t, J=7.3 Hz, 2H), 7.27 (t, J=7.3 Hz, 1H), 4.02 (m, 1H), 3.11 (m, 2H), 2.18 (m, 1H), 1.84 (m, 3H).

Compound 27:

N,N-Dimethylglycine (506 mg, 4.91 mmol) was suspended in CH₂Cl₂ (5 mL) and treated with oxalyl chloride (430 mL, 4.91 mmol) and 2 drops of DMF. After 1 hour the solution was warmed to room temperature and stirred for 1 hour. A THF (5 mL) solution of compound 3 (450 mg, 1.55 mmol) and triethylamine (1.37 mL, 9.83 mmol) was added and the resulting suspension was stirred over night. Water (10 mL) was added and the solid was filtered and washed with water (2×5 mL) and ethyl acetate (2×5 mL) to yield compound 27 (267 mg). ¹H NMR (200 MHz, DMSO-d⁶) δ 9.14 (br s, 1H), 8.70 (br s, 1H), 7.87 (m, 2H), 7.24 (m, 2H), 3.77 (s, 2H), 2.71 (s, 6H). MS (API-ES, positive scan, m/z) M+1=384.1.

Compound 28.

Compound 28 was prepared as per compounds 15 by treating compound 5 with acetic anhydride instead of acetyl chloride, and catalytic DMAP, to provide a yellow solid after triturating with MeOH. ¹H NMR (200 MHz, DMSO-d⁶) δ 8.79 (s, 1H), 7.48 (s, 1H), 7.45 (d, J=8.5 Hz, 1H), 6.99 (d, J=8.5 Hz, 1H), 4.14 (m, 4H), 2.09 (m, 2H), 2.01 (s, 3H). MS (API-ES, positive scan, m/z) M+1=395.1.

Compound 29.

Compound 29 was prepared as per compounds 15 by treating compound 5 with 2-methoxyacetyl chloride instead of acetyl chloride, and catalytic DMAP, Saturated aqueous NH₄Cl (5 mL) and ethyl acetate (20 mL) were added and the organic layer was washer with brine (2×10 mL), dried over anhydrous MgSO₄, filtered and the solvent removed under reduced pressure to provide compound 29 as an of white solid. ¹H NMR (200 MHz, DMSO-d₅) δ 8.74 (s, 1H), 7.47 (s, 1H), 7.46 (d, J=8.2 Hz, 1H), 6.99 (d, J=8.2 Hz, 1H), 4.13 (m, 4H), 3.90 (s, 2H), 2.23 (s, 3H), 2.10 (m, 2H).

Compound 30.

Compound 30 was prepared as per compounds 15 by treating compound 6 with acetic anhydride instead of acetyl chloride, and catalytic DMAP. Saturated aqueous NH₄Cl (5 mL) and ethyl acetate (20 mL) were added and the organic layer was washer with brine (2×10 mL), dried over anhydrous MgSO₄, filtered and the solvent removed under reduced pressure to provide a yellow solid. ¹H NMR (200 MHz, DMSO-d⁶) δ 1.95 (s, 3H), 3.14 (t, J=4.3 Hz, 4H), 3.73 (t, J=4.0 Hz, 4H), 6.98 (d, J=8.9 Hz, 2H), 7.75 (d, J=8.8 Hz, 2H), 8.65 (s, 1H).

Compound 33.

Compound 33 was prepared as per compounds 15 by treating compound 6 with butyric anhydride, and catalytic DMAP. Saturated aqueous NH₄Cl (5 mL) and ethyl acetate (20 mL) were added and the organic layer was washer with brine (2×10 mL), dried over anhydrous MgSO₄, filtered and the solvent removed under reduced pressure to provide a yellow solid. ¹H NMR (200 MHz, DMSO-d⁶) δ 8.79 (s, 1H), 7.48 (s, 1H), 7.46 (d, J=8.8 Hz, 1H), 7.00 (d, J=8.8 Hz, 1H), 4.13 (m, 4H), 2.25 (t, J=7.0 Hz, 2H), 2.15 (m, 2H), 1.48 (t, J=7.0 Hz, 1H), 1.45 (q, J=7.0 Hz, 2H), 0.80 (t, J=7.0 Hz, 3H).

Compound 34:

Compound 34 was prepared as per compound 15 to provide a white solid after triturating with MeOH. ¹H NMR (200 MHz, DMSO-d₆) δ 8.92 (s, 1H), 7.98 (d, J=8.2 Hz, 2H), 7.75 (d, J=8.2 Hz, 2H), 7.40-7.20 (m, 3H), 6.92 (d, J=6.5 Hz, 1H), 3.92 (s, 3H), 2.02 (s, 3H).

Compound 35:

Compound 34 was prepared as per compound 15, using 2-methoxyacetyl chloride in place of acetyl chloride, to provide a white solid after triturating with MeOH (95% yield). ¹H NMR (200 MHz, DMSO-d⁶) δ 8.89 (s, 1H), 7.96 (d, J=8.4 Hz, 2H), 7.75 (d, J=8.4 Hz, 2H), 7.32 (m, 3H), 6.93 (m, 1H), 3.91 (s, 2H), 3.82 (s, 3H), 3.24 (s, 3H).

Compound 37.

Compound 37 was prepared as per compounds 15 to provide a yellow solid after triturating with MeOH. ¹H NMR (200 MHz, DMSO-d⁶) δ 8.98 (s, 1H), 8.19-7.90 (m, 4H), 7.83 (d, J=8.5 Hz, 2H), 7.72 (m, 2H), 2.01 (s, 3H).

Compound 40:

Compound 40 was prepared as per compound 15, using butyric anhydride, to provide a white solid after triturating with MeOH. ¹H NMR (200 MHz, DMSO-d⁶) δ 8.99 (s, ₁H), 8.04 (m, 4H), 7.84 (d, J=8.3 Hz, 2H), 7.72 (m, 2H), 3.38 (t, J=7.3 Hz, 2H), 1.5 (m, 2H), 0.81 (t, J=7.7.4 Hz, 3H).

Compound 41:

Compound 11 (250 mg, 0.5 mmol), 2-chloro-1-methylpyridinium iodide (140 mg, 0.55 mmol) DMAP (10 mg), and triethylamine (252 μL, 1.81 mmol) were suspended in THF (10 mL). The 2-(2-methoxyethoxy)acetic acid (73 μL, 0.55 mmol) was added and the mixture was stirred at room temperature for 3 days. The solution was extracted using ethyl acetate and water. The solution was treated with 1M HCl (10 mL) and extracted with ethyl acetate (50 mL). The organic layer was separated, dried over anhydrous MgSO₄, filtered, and the solvent was removed under reduced pressure. The resulting solid was purified trituration with acetone (105 mg). ¹H NMR (200 MHz, DMSO-d₆) δ 8.79 (s, 1H), 8.01 (m, 4H), 7.81 (m, 2H), 7.05 (m, 2H), 3.79 (s, 2H), 3.50 (m, 2H), 3.42 (m, 2H), 3.20 (s, 3H).

Compound 42:

Compound 42 was prepared as per compound 41, using 2-[-2-(methoxyethoxy)ethoxy]acetic acid (93 mg, 0.6 mmol). Purification by triturating with methanol provide a light yellow solid. ¹H NMR (200 MHz, DMSO-d₆) δ 8.61 (s, 1H0, 8.0-7.92 (m, 4H), 7.78-7.66 (m, 4H), 3.72 (s, 3H), 3.72-3.58 (m, 4H), 3.53 (m, 2H), 3.29 (m, 2H).

Compound 43:

Compound 15 (2.20 g, 7.92 mmol) was suspended in THF (120 mL) and treated with Boc₂O (2.03 g, 9.3 mmol) and triethylamine (1.10 mL, 7.9 mmol). The solution was stirred for 36 hours. The solvent was removed under reduced pressure, and the resulting solid was partitioned between ethyl acetate (200 mL) and water (100 mL). The organic layer was washed with water (2×100 mL), 10% citric acid (50 mL), and water (2×50 mL), dried over anhydrous MgSO₄, filtered, and volatiles remover under reduced pressure to provide compound 43 as a yellow solid (5.90 g, 100% yield). ¹H NMR (200 MHz, DMSO-d₅) δ 9.00 (s, 1H), 8.03 (m, 4H), 7.85 (m, 2H), 7.72 (m, 2H), 1.35 (s, 9H).

Compound 44:

Compound 44 was prepared as per compound 15 to provide a white solid after triturating with MeOH. ¹H NMR (200 MHz, DMSO-d⁶) δ 8.73 (s, 1H), 7.88 (d, J=8.2 Hz, 2H), 7.49 (d, J=8.2 Hz, 2H), 7.08-7.02 (m, 4H), 1.90 (s, 3H).

Compound 45:

Compound 45 was prepared as per compound 15, using methoxyacetyl chloride, provide a white solid after triturating with MeOH. ¹H NMR (200 MHz, DMSO-D⁶) δ 8.77 (s, 1H), 7.90 (d, J=8.2 Hz, 2H), 7.42 (d, J=8.2 Hz, 2H), 7.06 (m, 4H), 3.89 (s, 2H), 3.22 (s, 3H).

Compound 46:

Compound 46 was prepared as per compound 15, using butyric anhydride, to provide a white solid after triturating with MeOH. ¹H NMR (200 MHz, DMSO-d₆) δ 8.80 (s, 1H), 7.92 (d, 8.0 Hz, 2H), 7.45 (d, J=8.0 Hz, 2H), 7.07 (m, 4H), 2.20 (t, 2H), 1.46 (q, 2H), 0.80 (t, 3H).

Compound 47:

Compound 47 was prepared as per compound 15, using pivavoyl chloride, to provide a white solid after triturating with MeOH. ¹H NMR (200 MHz, DMSO-d⁶) δ 8.74 (s, 1H), 7.89 (d, J=8.2 Hz, 2H), 7.42 (d, J=8.0 Hz, 2H), 7.05 (m, 4H), 2.13 (m, 2H), 1.41 (m, 4H), 1.25-1.08 (m, 22H), 0.82 (br t, 3H).

Compound 48:

Compound 13 (200 mg, 0.466 mmol) was suspended in tetrahydrofuran (THF) (10 mL) and treated with benzyl chloroformate (2.0 equiv) and triethylamine (2.5 equiv). The reaction mixture was heated to reflux and stirred overnight. The solution was treated with 1M HCl (10 mL) and extracted with ethyl acetate (50 mL). The organic layer was separated, dried over anhydrous MgSO₄, filtered, and the solvent was removed under reduced pressure. The resulting solid was purified by silica gel chromatography, using a 5-100% EtOAc/hexane gradient (FlashMaster Solo LC) to yield compound 48 as yellow powder (110 mg, 44%). ¹H NMR (200 MHz, DMSO-d₆) δ 8.67(s, 1H), 7.90(d, 2H, J=8.9 Hz), 7.42(d, 2H, J=8.9 Hz), 7.26(m, 5H), 7.07(q, 4H, J₁=2.1 Hz, J₂=8.9 Hz), 4.85(s, 2H).

Compound 49:

Compound 13 (300 mg, 0.7 mmol), 2-chloro-1-methylpyridinium iodide (1.5 equiv.), DMAP (0.15 equiv), and triethylamine (4 equiv) were suspended in THF (10 mL). The Boc-Val-OH (1.5 equiv.) was added and the mixture was heated to reflux for 40 minutes. The solution was extracted using ethyl acetate and water. The solution was treated with 1M HCl (10 mL) and extracted with ethyl acetate (50 mL). The organic layer was separated, dried over anhydrous MgSO₄, filtered, and the solvent was removed under reduced pressure. The resulting solid was purified by chromatography using silica gel chromatography, using a 5-100% EtOAc/hexane gradient (FlashMaster Solo LC) to yield compound 49 as a light brown solid (35 mg, 8% yield). ¹H NMR (200 MHz, DMSO-d₆) δ 8.67 (s, 1H), 7.90 (d, 2H, J=8.9 Hz), 7.43 (d, 2H, J=8.9 Hz), 7.06 (d, 4H, J=7.6 Hz), 5.97 (br d, 1H), 3.66 (m, 1H), 1.34(s, 9H), 0.78 (q, 6H, J₁=6.4 Hz, J₂=15.6 Hz)

Compound 50:

Compound 50 was prepared as per compound 49 using Boc-Phg-OH, to provide a yellow powder (118 mg, 26% yield). ¹H NMR (200 MHz, DMSO-d₆) δ 8.67 (s, 1H), 7.90 (d, 2H, J=8.5 Hz), 7.43 (d, 2H, J=9.2 Hz), 7.29 (m, 5H), 7.07 (m, 4H), 6.79 (br d, 1H), 4.90 (br d, 1H), 1.33(s, 9H)

Compound 51:

Compound 51 was prepared as per compound 49 using Boc-Arg-OH. The resulting solid was purified by chromatography using silica gel chromatography, using a 10-50% MeOH/CH₂Cl₂ gradient (FlashMaster Solo LC) to yield compound 51 as a light brown solid (15 mg, 3% yield). ¹H NMR (200 MHz, DMSO-d₆) δ 8.64 (s, 1H), 7.89 (d, 2H, J=8.5 Hz), 7.43 (d, 2H, J=8.5 Hz), 7.07 (q, 4H, J₁=2.6 Hz, J₂=8.7 Hz), 6.27 (d, 1H, J=8.2 Hz), 3.86 (m, 1H), 3.05 (m, 2H), 1.71 (br s, 1H), 1.46 (m, 4H), 1.35 (s, 9H).

Compound 53 to 140:

Compounds 53 to 140 were prepared as previously described (see PCT Application No. CA02/01942 (WO 03/051890)). Compounds 141 to 149 were prepared in a manner similar to that described in (PCT Application No. CA02/01942 (WO 03/051890)).

Compound 141:

¹H NMR (DMSO d⁶, 200 MHz) δ 8.95 (s, 1H), 8.73 (s, 2H), 8.32 (d, J=6.7 Hz, 1H), 8.02 (S, 1 h), 7.89 (d, J=7.3 Hz, 1H), 7.45-7.39 (m, 2H), 3.65 (d, J=6.7 Hz, 2H), 1.50-1.45 (m, 2H), 1.31-1.24 (m, 2H), 0.73 (t, J=7.0 Hz, 3H); ¹³C NMR (DMSO d⁶, 50 MHz) δ 163.9, 145.0, 140.3, 135.1, 127.1, 124.9, 123.7, 123.5, 121.6, 114.9, 113.0, 111.3.

Compound 142:

¹H NMR (DMSO d⁶, 200 MHz) δ 8.88 (s, 1H), 8.73 (s, 2H), 8.27 (d, J=6.4 Hz, 1H), 7.75 (s, 1H), 7.66 (d, J=7.9 Hz, 1H), 7.35-7.17 (m, 5H), 7.05 (s, 1H), 7.02 (d, J=6.7 Hz, 1H), 5.03 (s, 2H); ¹³C NMR (DMSO d⁶, 50 MHz) δ 164.0, 145.0, 140.4, 135.5, 130.8, 129.0, 128.5, 127.5, 126.8, 124.8, 124.0, 123.4, 121.4, 114.8, 113.1, 111.3, 58.9.

Compound 143:

¹H NMR (200 MHz, DMSO-d⁶) δ 8.95 (s, 1H), 8.75 (s, 2H), 8.26-8.21 (m, 4H), 8.03-7.93 (m, 3H), 7.45-7.38 (m, 2H).

Compound 144:

¹H NMR (DMSO d⁶, 200 MHz) δ 8.92 (s, 1H), 8.75 (s, 2H), 8.35-8.23 (m, 3H), 8.27 (s, 1H), 8.07-8.04 (m, 2H), 7.38 (t, J=7.9 Hz, 1H), 7.49-7.33 (m, 2H); ¹³C NMR (DMSO d⁶, 50 MHz) δ 164.3, 145.2, 139.9, 137.9, 134.7, 131.8, 130.9, 130.2, 127.8, 125.7, 124.4, 123.4, 121.9, 120.3, 117.3, 113.4, 111.9, 96.0.

Compound 145:

¹H NMR (200 MHz, DMSO-d⁶) δ 8.95 (s, 1H), 8.75 (s, 2H), 8.37-8.26 (m, 6H), 8.02 (d, J=7.3 Hz, 1H), 7.45-7.37 (m, 2H).

Compound 146:

¹H NMR (DMSO d⁶, 200 MHz) δ 8.93 (s, 1H), 8.75 (s, 2H), 8.24 (d, J=8.9 Hz, 1H), 8.22 (s, 1H), 8.14-8.07 (m, 2H), 8.01 (d, J=7.9 Hz, 1H), 7.46-7.37 (m, 4H).

Compound 147:

¹H NMR (200 MHz, DMSO-d⁶) δ 8.93 (s, 1H), 8.74 (s, 2H), 8.25-8.19 (m, 2H), 7.95-7.92 (m, 3H), 7.37-7.39 (m, 2H), 7.05 (d, J=8.2 Hz, 2H).

Compound 148:

¹H NMR (DMSO d⁶, 200 MHz) δ 8.94 (s, 1H), 8.77 (s, 2H), 8.32 (d, J=7.0 Hz, 1H), 8.02 (s, 1H), 7.88 (d, J=7.3 Hz, 1H), 7.46-7.37 (m, 2H), 3.49 (s, 3H); ¹³C NMR (DMSO d⁶, 50 MHz) δ 164.1, 145.1, 140.5, 134.9, 127.3, 125.1, 123.7, 123.6, 121.7, 115.2, 113.2, 111.5.

Compound 149:

¹H NMR (DMSO d⁶, 200 MHz) δ 8.99 (s, 1H), 8.74 (s, 2H), 8.17 (s, 1H), 7.89 (d, J=6.7 Hz, 1H), 7.61-7.24 (m, 5H), 6.94 (d, J=7.3 Hz, 1H), 3.82 (s, 3H); ¹³C NMR (DMSO d⁶, 50 MHz) δ 189.3, 184.9, 171.7, 170.3, 166.5, 165.7, 158.9, 155.1, 154.5, 151.4, 149.2, 148.4, 144.2, 138.2, 137.4, 136.5.

Compound 150:

Compound 1 (1.50 g, 4.15 mmol) was dissolved in THF (180 mL) and treated with triethylamine (2.52 mL, 24.9 mmol) and sebacoyl chloride (2.98 g, 12.4 mmol). This mixture was stirred for 2 hours prior to the addition of PEG 400 (5.32 g, 13.2 mmol). The colution was stirred an addition hour before 1M HCl (20 mL) and ethyl acetate (10 mL) were added. The organic layer was washed with water (2×50 mL), dried over anhydrous MgSO4, filtered, and the volatiles removed under reduced pressure. The resulting semi-solid was dissolved in a minimum amount of methanol and purified by C18 reverse phase chromatography, eluting with a 5-100% acetonitrile water gradient, to provide compound 150 as a yellow semi-solid. ¹H NMR (200 MHz, CD₃OD) δ 8.52 (s, 1H), 7.83 (m, 2H), 7.49-7.30 (m, 3H), 4.17 (m, 1H), 3.63 (m, 14H), 2.40-2.20 (m, 4H), 1.58 (m, 5H), 1.40-1.20 (m, 9H).

Example: Rat Model of Cisplatin Induced Neuropathy

Male Sprague-Dawley rats (weighing 200-225 g on arrival) were intraperitoneally administered 2.5 mg/kg cisplatin daily, for five consecutive days to achieve a final cumulative dose of 12.5 mg/kg. On the third day following the final cisplatin injection, animals received compounds SC at concentrations of (3, 10, and 30 mg/kg). Dosing continued Monday through Friday for three consecutive weeks.

The effect of cisplatin on peripheral nerve function, and the ability of the compounds to attenuate the cisplatin effect were determined after three weeks of drug treatment by measuring the sensory nerve conduction velocity (SNCV) in the caudal nerve of the tail. Stimulating electrodes were used to deliver 2 mA pulses once per second for 1.5 min. The resulting compound sensory nerve action potentials were averaged, and the mean response onset time was determined from the averaged response. Two mean response times were determined, the second being 20 mm distal from the first. The difference in onset time between the two recordings was determined and used to calculate the conductance velocity.

$$SNCV = \frac{distance ( 20 mm )}{Distal onset - Proximal Onset ( msec )}$$

The results of these experiments were combined, and ANOVA was performed, followed by a Fisher LSD test.

Example: Anti-Cancer Activity

Compounds were tested for anti-cancer properties using the Alamar blue viability assay. Daoy human medulloblastoma cells of 15N neuroblastoma cells were plated at a density of 5000 cells per well of a 96 well plate and cultured in RPMI media supplemented with antibiotics and 5% fetal bovine serum. Cells in culture were incubated with compound for 48 hours after which time Alamar blue was added to the culture media. After 4 hours media was transferred to opaque white plates and fluorescence of transformed alamar blue was measured at excitation 535/emission 595). A1 resulted in a dose dependent decrease in medulloblastoma cell viability.

Example: Clonogenic Assays

Du145 prostate, HCT116 colon, 15N Neuroblastoma, IMR32 Neuroblastoma, Daoy Medulloblastoma, and MDAMB231 breast cells were plated in 6 well plates and allowed to grow for 5 days._Cells were exposed to compound for 24 hours, the culture media was removed and replaced with fresh media. The cells were kept in cultured for 7-10 days after which colonies were counted and EC₅₀ values were determined relative to non-treated controls.

1. A compound represented by Formula I: 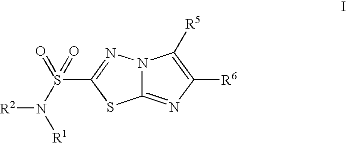
or a pharmaceutically acceptable salt thereof, wherein:
R¹ is selected from the group consisting of:
a) C(O)R⁹, wherein R⁹ is selected from substituted or unsubstituted C(1-18) alkyl, substituted or unsubstituted aryl, substituted or unsubstituted heteroaryl;
b) C(O)—(CH₂)ₙ—(C(O))ₚ—(OCH₂CH₂)ₘOR¹⁰, wherein n=0-6, p=0-1, m=0-22; and R¹⁰ is H, substituted or unsubstituted C(1-6) alkyl, substituted or unsubstituted aryl, substituted or unsubstituted heteroaryl; and
c) C(O)—(CHR¹¹)ₙ—NR¹²R¹³ wherein n=1-5; and R¹¹ is selected from the group consisting of: hydrogen, substituted or unsubstituted C(1-8) alkyl, substituted or unsubstituted C(1-8) aralkyl, substituted or unsubstituted C(1-8) aryl, and substituted or unsubstituted C(1-8) heteroaryl; and wherein R¹² and R¹³ are individually selected from the group consisting of: hydrogen, substituted or unsubstituted C(1-8) alkyl, substituted or unsubstituted C(1-8) aralkyl, substituted or unsubstituted C(1-8) aryl, substituted or unsubstituted C(1-8) heteroaryl, substituted or unsubstituted C(1-8) alkylcarbonyl, substituted or unsubstituted C(1-8) arylcarbonyl, and substituted or unsubstituted C(1-8) heteroarylcarbonyl; or wherein R¹² and R¹³ are combined to form a 5 to 7 membered substituted or unsubstituted heterocyclic ring system;
R² is H or C(1-4) alkyl;
R⁵ is selected from the group consisting of: H, methyl, and substituted or unsubstituted benzyl;
R⁶ is selected from the group consisting of:
(i) fluoro C(1-6)-alkyl, substituted and unsubstituted C(6-16)-aryl, substituted and unsubstituted heteroaryl, substituted and unsubstituted coumarinyl, and adamantyl; 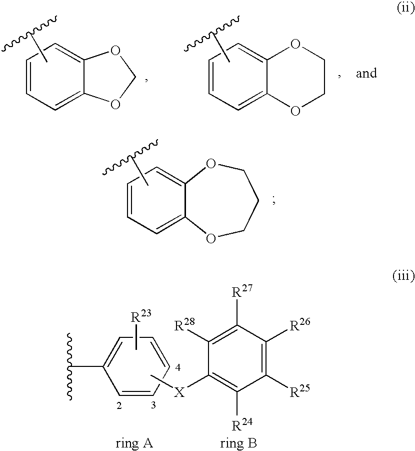
wherein
X is represented by a bond, O or S(O)ₙ, wherein n=0, 1, or 2, and is attached to ring A at the 2, 3, or 4 position;
R²³ on ring A is selected from the group consisting of H, halogen, C(1-8)alkyl, C(1-8) alkoxy and represents up to 4 substitutions;
R²⁴ through R²⁸ of ring B is independently selected from the group consisting of: H, halogen, C(1-8) alkyl, C(1-8) fluoroalkyl, C(1-8) alkoxy,
wherein any two adjacent R²⁴ through R²⁸ groups may be combined to form a fused aryl, substituted aryl, heteroaryl, or substituted heteroaryl ring system; and 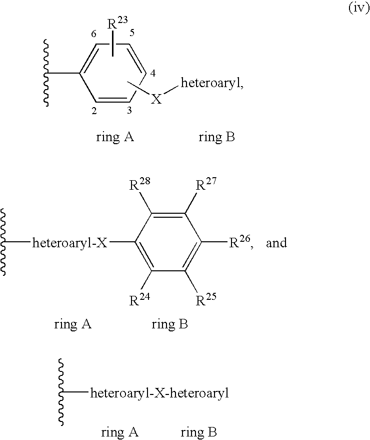
wherein
X is represented by a bond, O or S(O)ₙ, wherein n=0, 1, or 2;
R²³ on ring A is selected from the group consisting of: H, halogen, C(1-8) alkyl, C(1-8) alkoxy and represents up to 4 substitutions;
R²⁴ through R²⁸ of ring B are independently selected from the group consisting of: H, halogen, C(1-8) alkyl, C(1-8) fluoroalkyl, and C(1-8) alkoxy; and wherein any two adjacent R²⁴ through R²⁸ groups may be combined to form a fused aryl, substituted aryl, heteroaryl, or substituted heteroaryl ring system; and
wherein the heteroaryl ring systems of ring A and B contain at least one heteroatom and are substituted or unsubstituted.

2. The compound, according to claim 1, in which R¹ is C(O)R⁹, wherein R⁹ is selected from substituted or unsubstituted alkyl C(1-18).

3. The compound, according to claim 2, in which R¹ is C(O)R⁹, wherein R⁹ is substituted or unsubstituted alkyl C(1-8) alkyl.

4. The compound, according to claim 1, in which R¹ is C(O)—(CH₂)ₙ—(C(O))ₚ—(OCH₂CH₂)ₘOR¹⁰, wherein n=0-6, p=O-1, m=0-22; and R¹⁰ is H, substituted or unsubstituted C(1-6) alkyl.

5. The compound according to claim 4, in which R¹⁰ is H or CH₃.

6. The compound, according to claim 1, in which R¹ is C(O)—(CHR¹¹)ₙ—NR¹²R¹³ wherein n=1-5; R¹¹ is selected from the group consisting of: hydrogen, substituted or unsubstituted C(1-8) alkyl, substituted or unsubstituted C(1-8) aralkyl, substituted or unsubstituted C(1-8) aryl, substituted or unsubstituted C(1-8) heteroaryl; and R¹² and R¹³ are individually selected from the group consisting of: hydrogen, substituted or unsubstituted C(1-8) alkyl, substituted or unsubstituted C(1-8) aralkyl, substituted or unsubstituted C(1-8) aryl, substituted or unsubstituted C(1-8) heteroaryl, substituted or unsubstituted C(1-8) alkylcarbonyl, substituted or unsubstituted C(1-8) arylcarbonyl, substituted or unsubstituted C(1-8) heteroarylcarbonyl; or R¹² and R¹³ are combined to form a 5 or 6 membered substituted or unsubstituted heterocyclic ring system.

7. The compound, according to claim 6, in which n=1.

8. The compound, according to claim 6, in which R¹¹ is selected from hydrogen, substituted or unsubstituted C(1-8) alkyl.

9. The compound, according to claim 6, in which R¹² and R¹³ are individually selected from hydrogen and substituted or unsubstituted C(1-8) alkyl.

10. The compound, according to claim 6, in which R¹² and R¹³ are combined to form a 5 or 6 membered substituted or unsubstituted heterocyclic ring system.

11. The compound, according to claim 1, in which R² is H or methyl.

12. The compound, according to claim 11, in which R² is H.

13. The compound, according to claim 1, in which R⁵ is H.

14. The compound, according to claim 1, in which R⁶ is selected from the group consisting of:
(i) fluoro C(1-6)-alkyl, substituted and unsubstituted C(6-16)-aryl, substituted and unsubstituted heteroaryl, substituted and unsubstituted coumarinyl, and adamantyl; 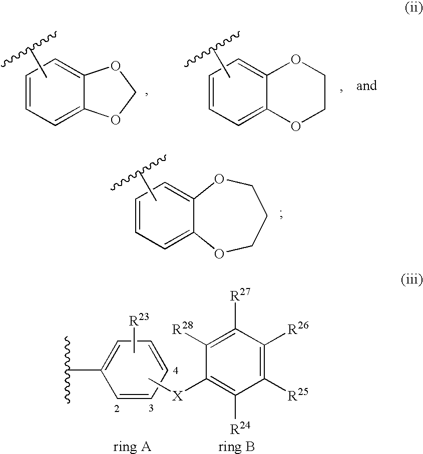
wherein
X is represented by a bond, O or S(O)ₙ, wherein n=0, 1, or 2, and is attached to ring A at the 2, 3, or 4 position;
R²³ on ring A is selected from the group consisting of H, halogen, C(1-8)alkyl, C(1-8) alkoxy and represents up to 4 substitutions;
R²⁴ through R²⁸ of ring B is independently selected from the group consisting of: H, halogen, C(1-8) alkyl, C(1-8) fluoroalkyl, C(1-8) alkoxy,
wherein any two adjacent R²⁴ through R²⁸ groups may be combined to form a fused aryl, substituted aryl, heteroaryl, or substituted heteroaryl ring system.

15. The compound, according to claim 12, in which R⁶ is selected from the group consisting of:
(i) substituted and unsubstituted C(6-16)-aryl, substituted and unsubstituted heteroaryl; 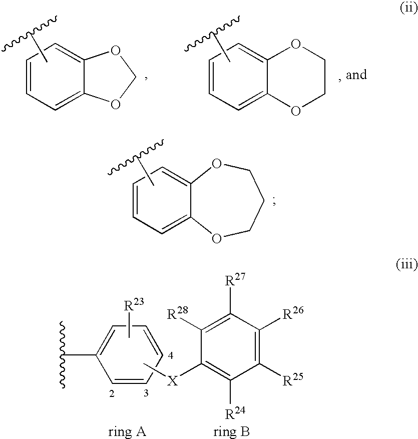
wherein
X is represented by a bond, 0, and is attached to ring A at the 2, 3, or 4 position;
R²³ on ring A is hydrogen; and
R²⁴ through R²⁸ of ring B is independently selected from the group consisting of: H, halogen, C(1-8) alkyl, C(1-8) fluoroalkyl and C(1-8) alkoxy.

16. The compound, according to claim 1, in which R⁶ is chosen from the following:

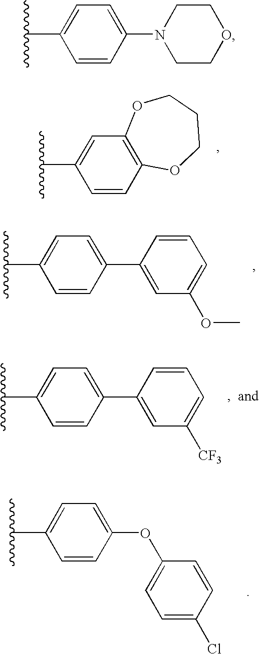

17. The compound, according to claim 1, in which the substituents are selected from the group consisting of:
1) H, halogen, nitro, cyano, C(1-8) alkyl, C(1-8) fluoroalkyl, aralkyl, aryl, heteroaryl, C(1-8) alkylcarbonyl, arylcarbonyl, heteroarylcarbonyl, azide, B(OH)₂, and adamantyl;
2) XR¹⁹ wherein X═O or S and R¹⁹ is C(1-8) alkyl, hydroxyl, C(1-4) alkoxy, fluoroalkyl, aryl, heteroaryl, lower alkylcarbonyl, arylcarbonyl, heteroarylcarbonyl, lower alkylaminocarbonyl, and arylaminocarbonyl; and
3) NR¹⁴R¹⁵ wherein R¹⁴ and R¹⁵ are each independently C(1-8) alkyl, or wherein R¹⁴ and R¹⁵ are joined to form an alkyl or heteroalkyl ring system,
wherein the C(1-8) alkyl, C(1-8) fluoroalkyl, aralkyl, aryl, heteroaryl, C(1-8) alkylcarbonyl, arylcarbonyl, heteroarylcarbonyl, and C(1-4) alkoxy may be further substituted by the substituents from 1), 2), and 3) above.

18. A compound represented by Formula I 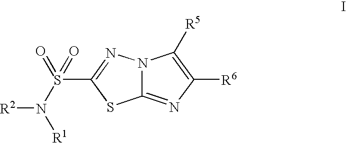
group consisting of:

| Com- | | | | |
|---|---|---|---|---|
| pound | R¹ | R² | R⁵ | R⁶ |
| 15 | CH₃C(O)— | H | H | Ph; |
| 16 | CH₃CH₂CH₂C(O)— | H | H | Ph; |
| 17 | tert-BuOC(O)— | H | H | Ph; |
| 18 | Boc(H)NCH₂C(O)— | H | H | Ph; |
| 19 | TFA.H₂NCH₂C(O)— | H | H | Ph; |
| 20 | Ac(H)NCH₂C(O)— | H | H | Ph; |
| 21 | 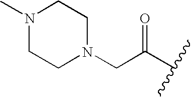 | H | H | Ph; |
| 22 | HO₂CCH₂CH₂C(O)— | H | H | Ph; |
| 23 | 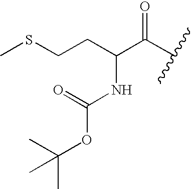 | H | H | Ph; |
| 24 | 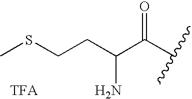 | H | H | Ph; |
| 25 | 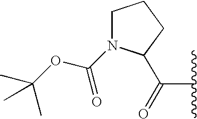 | H | H | Ph; |
| 26 | 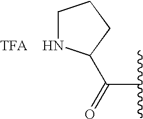 | H | H | Ph; |
| 27 | (CH3)2NCH2C(0)- | H | H | 4′-F—Ph; |
| 28 | CH₃C(O)— | H | H | diox-Ph; |
| 29 | CH₃OCH₂C(O)— | H | H | diox-Ph; |
| 30 | CH₃CH₂CH₂C(O)— | H | H | diox-Ph; |
| 31 | CH₃C(O)— | H | H | 4-morph-Ph; |
| 32 | CH₃OCH₂C(O)— | H | H | 4-morph-Ph; |
| 33 | CH₃CH₂CH₂C(O)— | H | H | 4-morph-Ph; |
| 34 | CH₃C(O)— | H | H | 3′-MeO-biPh; |
| 35 | CH₃OCH₂C(O)— | H | H | 3′-MeO-biPh; |
| 36 | CH₃CH₂CH₂C(O)— | H | H | 3′-MeO-biPh; |
| 37 | CH₃C(O)— | H | H | 3′-CF₃-biPh; |
| 38 | CH₃CH₂CH₂C(O)— | H | H | 3′-CF₃-biPh; |
| 39 | CH₃OCH₂C(O)— | H | H | 3′-CF_{3-biPh;} |
| 40 | CH₃CH₂CH₂C(O)— | H | H | 3′-CF₃-biPh; |
| 41 | 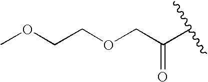 | H | H | 3′-CF₃-biPh; |
| 42 | 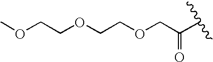 | H | H | 3′-CF₃-biPh; |
| 43 | tert-BuOC(O)— | H | H | 3′-CF_{3-biPh;} |
| 44 | CH₃C(O)— | H | H | 4-(4-Cl—PhO)Ph; |
| 45 | CH₃OCH₂C(O)— | H | H | 4-(4-Cl—PhO)Ph; |
| 46 | CH₃CH₂CH₂C(O)— | H | H | 4-(4-Cl—PhO)Ph; |
| 47 | 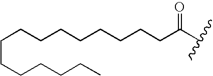 | H | H | 4-(4-Cl—PhO)Ph; |
| 48 | PhCH₂OC(O)— | H | H | 4-(4-Cl—PhO)Ph; |
| 49 | 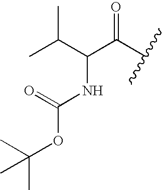 | H | H | 4-(4-Cl—PhO)Ph; |
| 50 | 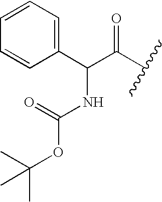 | H | H | 4-(4-Cl—PhO)Ph; and |
| 51 | 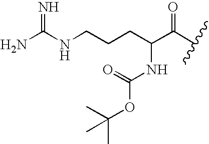 | H | H | 4-(4-Cl—PhO)Ph. |

and

19. A compound, according to the following formula:

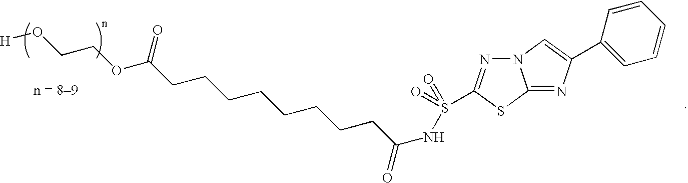

20. The compound, according to claim 1, is a salt encapsulated in an encapsulating agent.

21. The compound according to claim 20, wherein the encapsulating agent is a cyclodextran.

22. The compound according to claims 20, wherein the encapsulating agent is hydroxypropylcyclodextran (HPCD).

23. The compound, according to claim 20, in which the salt is selected from the group consisting of: a sodium salt, an ethanolamine salt, a dimethylaminoethanol salt, and a 4-aminopyridine salt.

24. The compound according to claim 23, in which the salt is a sodium salt.

25. The compound, according to claim 1, is a prodrug.

26. A prodrug represented by Formula I: 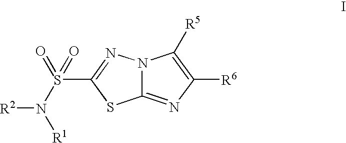
or a pharmaceutically acceptable salt thereof, wherein:
R¹ is selected from the group consisting of:
d) C(O)R⁹, wherein R⁹ is selected from substituted or unsubstituted C(1-18) alkyl, substituted or unsubstituted aryl, substituted or unsubstituted heteroaryl;
e) C(O)—(CH₂)ₙ—(C(O))ₚ—(OCH₂CH₂)ₘOR¹⁰, wherein n=0-6, p=0-1, m=0-22; and R¹⁰ is H, substituted or unsubstituted C(1-6) alkyl, substituted or unsubstituted aryl, substituted or unsubstituted heteroaryl; and
f) C(O)—(CHR¹¹)ₙ—NR¹²R¹³ wherein n=1-5; and R¹¹ is selected from the group consisting of: hydrogen, substituted or unsubstituted C(1-8) alkyl, substituted or unsubstituted C(1-8) aralkyl, substituted or unsubstituted C(1-8) aryl, and substituted or unsubstituted C(1-8) heteroaryl; and wherein R¹² and R¹³ are individually selected from the group consisting of: hydrogen, substituted or unsubstituted C(1-8), alkyl, substituted or unsubstituted C(1-8) aralkyl, substituted or unsubstituted C(1-8) aryl, substituted or unsubstituted C(1-8) heteroaryl, substituted or unsubstituted C(1-8) alkylcarbonyl, substituted or unsubstituted C(1-8) arylcarbonyl, and substituted or unsubstituted C(1-8) heteroarylcarbonyl; or wherein R¹² and R¹³ are combined to form a 5 to 7 membered substituted or unsubstituted heterocyclic ring system;
R² is H or C(1-4) alkyl;
R⁵ is selected from the group consisting of: H, methyl, and substituted or unsubstituted benzyl;
R⁶ is selected from the group consisting of:
(i) fluoro C(1-6)-alkyl, substituted and unsubstituted C(6-16)-aryl, substituted and unsubstituted heteroaryl, substituted and unsubstituted coumarinyl, and adamantyl; 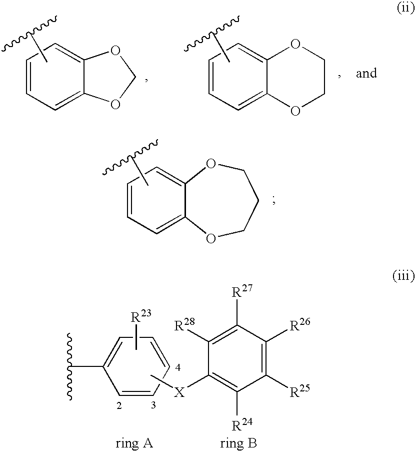
wherein
X is represented by a bond, O or S(O)ₙ, wherein n=0, 1, or 2, and is attached to ring A at the 2, 3, or 4 position;
R²³ on ring A is selected from the group consisting of H, halogen, C(1-8)alkyl, C(1-8) alkoxy and represents up to 4 substitutions;
R²⁴ through R²⁸ of ring B is independently selected from the group consisting of: H, halogen, C(1-8) alkyl, C(1-8) fluoroalkyl, C(1-8) alkoxy,
wherein any two adjacent R²⁴ through R²⁸ groups may be combined to form a fused aryl, substituted aryl, heteroaryl, or substituted heteroaryl ring system; and 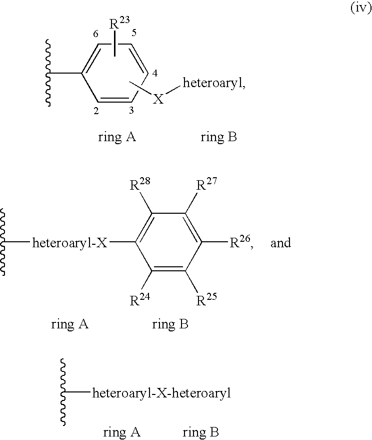
wherein
X is represented by a bond, O or S(O)ₙ, wherein n=0, 1, or 2;
R²³ on ring A is selected from the group consisting of: H, halogen, C(1-8) alkyl, C(1-8) alkoxy and represents up to 4 substitutions;
R²⁴ through R²⁸ of ring B are independently selected from the group consisting of: H, halogen, C(1-8) alkyl, C(1-8) fluoroalkyl, and C(1-8) alkoxy; and wherein any two adjacent R²⁴ through R²⁸ groups may be combined to form a fused aryl, substituted aryl, heteroaryl, or substituted heteroaryl ring system; and
wherein the heteroaryl ring systems of ring A and B contain at least one heteroatom and are substituted or unsubstituted,
wherein the prodrug is convertible in vivo or in vitro to a compound represented by Formula I-a 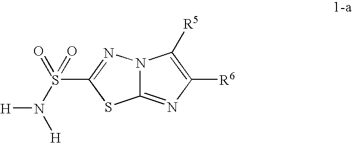
wherein R⁵ and R⁶ are as defined above.

27. A composition comprising a compound, according to claim 1, together with a carrier.

28. A method of preventing or treating peripheral neuropathy in a subject, the method comprising administering to the subject in need thereof an effective amount of the composition, according to claim 27.

29. The method, according to claim 28, in which the peripheral neuropathy is induced by a toxic agent.

30. The method, according to claim 29, in which the toxic agent is a neurotoxic agent or a chemotherapeutic agent.

31. The method, according to claim 30, in which the chemotherapeutic agent is dideoxyinosine, deoxy cytizine, D4T, cisplatin, etoposide, vincristine, epithilone or its derivatives, Taxol™/Taxoter™ or derivatives thereof.

31. The method, according to claim 30, in which the neurotoxic agent is vincristine, vinblastine, cisplatin, Taxol™, D4T or other antivirals, dideoxy compounds, alcohol, metals, industrial toxins, overdoses of vitamins A, D or B6, penicillin or chloramphenicol.

32. A method of treating a neurodegenerative disease in a subject, the method comprising administering the subject in need thereof an effective amount of the composition, according to claim 27.

33. The method, according to claim 32, in which the neurodegenerative disease is Alzheimer's disease, Parkinson's disease, ALS, Huntington's disease, muscular dystrophy, diabetes, HIV, an ischemic insult, retinal ganglion loss following acute ocular stroke or glaucoma, a neurodegenerative condition resulting from a viral infection, and a neuropathy resulting from the use of chemotherapeutic agents used in the treatment of HIV.

34. The method, according to claim, 32 in which the neurodegenerative disease is a degenerative disease of the eye.

35. A method of treating a neurodegenerative disease in a subject, the method comprising co-administering to the subject in need thereof the composition, according to claim 27, with COX-2 inhibitors, NSAIDS, acetylcholinesterase inhibitors, L-dopa, ACE inhibitors or insulin.

36. A method of inducing axonal growth and/or repair in a subject, the method comprising administering the subject in need thereof an effective amount of the composition, according to claim 27

37. A method of inducing axonal growth and/or repair in a subject, the method comprising administering to the subject in need thereof the composition, according to claim 27.

38. A method of altering signal transduction in a subject, the method comprising administering to the subject in need thereof the composition, according to claim 27.

39. A method of treating a proliferative disease in a subject, the method comprising administering to the subject in need thereof an effective amount of the composition, according to claim 27.

40. The method, according to claim 39, in which the proliferative condition is cancer.

41. The method, according to claim 40, in which the cancer is selected from the group consisting of prostate, colon, neuroblastoma, medulloblastoma, and breast cancer.

42. A method of treating a proliferative disease in a subject, the method comprising co-administering to the subject in need thereof the composition, according to claim 27, with a chemotherapeutic.

43. The method, according to claim 42, in which the chemotherapeutic is Taxol, cisplatin or vinca alkaloids.

44. A process for producing a compound of Formula 1, according to claim 1, the process comprising:
coupling the sulfonamide: 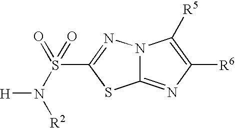
and either R⁹COCl or (R⁹CO)₂O in a solvent and a base so as to produce the following: 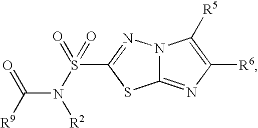
wherein R², R⁵, R⁶, and R⁹ are as defined herein.

45. A process for producing a compound of Formula I, according to claim 1, the process comprising:
coupling the sulfonamide: 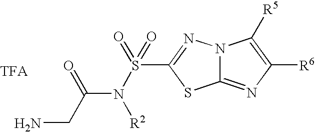
and either R⁹COCl or (R⁹CO)₂O in a solvent and a base so as to produce the following: 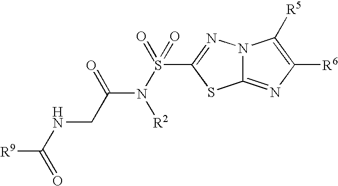
wherein R², R⁵, R⁶, and R⁹ are as defined herein.
